(19) United States
(12) Patent Application Publication
(10) Pub. No.: US 2005/0142534 A1
(43) Pub. Date: Jun. 30, 2005

(54) Methods and compositions related to a matrix chip

(75) Inventors: Andrew J. Maniotis, Berwyn, IL (US); Robert Folberg, Northbrook, IL (US)

Correspondence Address: FULBRIGHT & JAWORSKI L.L.P., 600 CONGRESS AVE., SUITE 2400, AUSTIN, TX, 78701, US

(73) Assignee: The Board of Trustees of the University of Illinois

(21) Appl. No.: 10/963,921

(22) Filed: Oct. 13, 2004

Related U.S. Application Data
(60) Provisional application No. 60/511,543, filed on Oct. 14, 2003.
(60) Provisional application No. 60/526,792, filed on Dec. 4, 2003.
(60) Provisional application No. 60/574,437, filed on May 26, 2004.

(51) Int. Cl. A01N 1/00; C12Q 1/68

(52) U.S. Cl. 435/4; 435/6

(57) ABSTRACT

Embodiments of the invention relate to devices and methods for evaluating the interactions between cells and between cells and matrix materials wherein the cellular distribution patterns formed as a result of such interactions are indicators of the invasive potential(s) of the cells. Furthermore, such devices and methods can provide indications of the preferred sites of metastasis of invasive cells; the efficacy of an anti-cancer drug applied to such cells; and the potential for agents to promote or enhance tumor growth or metastasis.

This application claims priority to U.S. Provisional Patent applications Ser. Nos. 60/511,543, filed Oct. 14, 2003; 60/526,792, filed Dec. 4, 2003; and 60/574,437, filed May 26, 2004; which are incorporated herein by reference in their entirety.

The government may own rights in the present invention pursuant to grant number ROI EY 10457 from the National Institutes of Health and grant number W-7405-ENG-48 from the Department of Energy.

### BACKGROUND

1. Field of the Invention

The invention relates generally to cell biology and cancer diagnosis. In particular, the invention relates to compositions, methods, and devices for detecting invasive mammalian cells, for differentiating between degrees of invasiveness of cells and for identifying compounds that regulate the invasiveness of cells.

2. Description of Related Art

Numerous methods have been devised for the detection of cancer. These range from the imaging of tumor masses by X-ray and optical techniques through the evaluation of cells in tissue samples obtained via biopsy to the detection of proteins and other molecular species that are released by cancerous cells into bodily fluids such as blood and urine. Of these methods, only the direct evaluation of cells obtained by biopsy is definitive for the detection, localization, and characterization of cancers and is thus the method of choice for such purposes either as a stand-alone method or as a means of confirming and elaborating upon results obtained through the use of other methods.

The cell-level detection, diagnosis, classification, and characterization of cancers have traditionally been carried out through the visual microscopic evaluation of the morphologies of the cells comprising a tissue or cellular specimen. More recently, automated methods of image analysis and immunohistochemical methods for the detection and quantitation of certain cell surface proteins (markers) that are specifically or differentially expressed by cancerous cells have come into use for this purpose. Proteomic techniques that identify cancer cells by evaluating changes in the expression of large suites of proteins are under development, but are not yet in routine clinical use. Although these newer techniques have certain utility in the detection of cancerous cells, the visual microscopic evaluation of cell morphology remains the accepted standard for the confirmation of such detection as well as for the diagnosis, classification and characterization of the cancer.

The primary limitation on the use of cell morphology for cancer detection largely derives from the low signal to noise ratio (SNR) that is inherent in this process. Cancer cells are generally a minor constituent of a clinical specimen, if present at all. For example, in the widely used “Pap smear” screening test for cervical cancer the presence of between one and ten abnormal cells within the population of roughly 50,000 to 300,000 cells that comprise a typical specimen is usually sufficient for the specimen to be declared to be positive for presence of cancer. Thus the signal to noise ratio for a positive specimen can be as low as 1/300,000 on this basis alone. This signal to noise ratio is further degraded by other factors, most notably “biological noise.”

Biological noise in such specimens derives from two primary sources. One source is the intrinsic variability between individual patients that reflects numerous genetic and environmental factors including, but not limited to medical history, demographics, and hormonal status. The other source derives from the fact that the morphological features of cells span a continuum from normal to overt cancer and do not present a clear-cut threshold for differentiating between cancerous and non-cancerous cells. Indeed, many morphological features associated with cancerous cells closely resemble, if not recapitulate those seen as a result of normal cellular processes such as repair of an injury or reaction to events such as infections. The relevant morphological criteria also vary somewhat between the various types of cancers. Factors such as these make cancer detection through the evaluation of cell morphology somewhat subjective and necessitate that such evaluations be performed by specially trained, highly skilled individuals. Despite such training and numerous attempts to systematize the evaluation of cell morphology, it is still often difficult to reach a consensus as to whether a particular cell is actually cancerous and, if so, the type of cancer it represents and its prognosis. As may be expected, this subjectivity also imposes significant limitations on the performance of automated systems for the evaluation of cell morphology.

One of the major driving forces behind the development of immunohistochemical and proteomic techniques for the detection of cancer has been the need to improve the signal to noise ratio of the detection process in order to improve the sensitivity and specificity of detection. The intent in immunohistochemical methods is to facilitate the differentiation between normal and cancerous cells by highlighting those cells that exhibit cell surface markers that are unique to, or are over expressed by a particular type of cancer. Such highlighted cells are then subjected to morphological evaluation to confirm the detection and to begin the process of classifying and characterizing the cancer. Proteomic methods employ the statistical and/or parametric interpretation of large suites of “indicators,” none of, which is necessarily definitive in and of itself, to similar purpose. Both methods, however, are known to be impacted by biological noise and, as a consequence, are used in conjunction with morphological analysis in order to obtain a definitive determination.

Economic and demographic factors are driving worldwide changes to the “standards of care” as they relate to cancer detection. The traditional emphasis has been on the use of highly sensitive screening tests to detect cancers at the earliest possible stage. Such highly sensitive tests intrinsically generate a high level of false positive results, each of which requires extensive and expensive follow-up and, therefore, represents a significant waste of health care resources. Epidemiological and other studies are now showing that, at least for certain cancers, it is more medically and economically effective to utilize tests that are highly specific and preferably prognostic to triage a screening -population into groups that are clearly normal, clearly cancerous, and “suspect.” In this model, follow-up and treatment are focused on those patients that are clearly shown to have cancer while patients in the suspect group receive an increased level of surveillance. This, in turn, permits redirecting the resources traditionally expended on resolving false positive test results into increasing the breadth and frequency of screening. At least in the area of cervical cancer screening where this movement is furthest advanced, this approach has been clearly demonstrated to improve the quality and availability of care in an economically viable manner. This is particularly important given increasing populations, an increasing average age of these populations and a continuing decline in the number of people who are trained in the morphological evaluation of cells.

For these and other reasons there is a need for an unambiguous and cost effective method for the detection of cancer cells that is sensitive, highly specific, generally applicable to a broad range of cancer types, and require minimal user skill and interpretation in order to arrive at a clinically useful conclusion. It is also desirable that the method be prognostic in that it determines the invasive potential of a detected cancer.

### SUMMARY OF THE INVENTION

Embodiments of the invention relate to devices and methods for evaluating the interactions between cells, and/or between cells and cellular matrix materials. The cellular distribution patterns formed because of such interactions can be indicative of the invasive potential of a particular cell or cells. Furthermore, such devices and methods can provide indications of the preferred sites of metastasis of invasive cells; the efficacy of an anti-cancer drug applied to such cells; and the potential for agents to promote or enhance tumor growth or metastasis.

The present invention is based upon the elucidation of the interactions between cells of various types and characteristics with different types and thicknesses of extracellular matrix and/or various matrix materials. Embodiments of the invention include devices and methods that provide for cancer detection, diagnosis, and characterization, as well as the evaluation of the efficacy of anti-cancer drugs and treatments; the evaluation of potential cancer promoting and enhancing agents; and in the study of cell growth, differentiation and gene expression. Specifically, the present invention is comprised of devices that permit the evaluation of cell growth and cell morphology as a function of the nature and thickness of an underlying layer comprising protein and/or other matrix materials and methods for the use of these devices for the purposes of cancer detection, diagnosis and characterization; drug discovery and evaluation; screening of agents for cancer promoting or enhancing activities; and research and investigation in the area of cell biology.

Embodiments of the invention include devices for evaluating cells comprising a substrate having one or more cellular growth regions comprised of a matrix material, wherein the matrix material within a region is: a) of a thickness A at which the cells to be evaluated do not penetrate and remodel the matrix material of the region; b) of a thickness B at which the cells to be evaluated can penetrate and remodel the matrix material of the region, but which does not permit the cells to become embedded in the material; c) of a thickness C at which the cells to be evaluated can penetrate, remodel and become embedded in the matrix material of the region; or d) a combination thereof. In certain aspects of the invention, the thickness A is less than 50 microns, thickness B between 50 and 100 microns, and thickness C greater than 100 microns. The matrix material may be comprised of extracellular proteins and/or other matrix materials. In certain embodiments, the matrix material may comprise proteinaceous components such as laminin, collagen, fibrinogen, fibronectin, cellular matrix material isolated from one or more biological tissues or combination thereof. One or more cellular growth regions of the device can be formed by painting, tampo printing, transfer printing, screen printing, ink jet deposition, lift off method of lithography, embossing, soft lithography, molding, casting, etch and fill (damascene), or combinations thereof. One or more cellular growth regions may be formed on a surface of or within cavities in a substrate or support material. Cells to be evaluated by the methods may be human cells, in particular human cell with the potential to be cancerous or pathologically hyperproliferative. The device may further comprise one or more reference regions of fibronectin as a reference against which patterns formed by cell growth on cellular growth regions comprising other matrix materials are compared.

In certain embodiments, a device for evaluating cells comprises a substrate having one or more cellular growth regions comprising a matrix material, wherein one or more cellular growth regions vary in thickness. In various embodiments, the one or more cellular growth regions vary in thickness in a continuous manner. In certain aspects of the invention, the thickness of one or more cellular growth regions vary from a minimum thickness that is (i) sufficient to permit cells to adhere to and grow on the matrix material, and (ii) is insufficient to permit the cells to penetrate and remodel the matrix material of the region to a maximum thickness that is sufficient to permit the cells to penetrate, remodel, and embedded in the matrix material of the region. The thickness of a cellular growth region may vary from less than 50 microns to greater than 500 microns or more. In certain preferred embodiments, the matrix material comprising the cellular growth region is laminin, collagen, fibrinogen, fibronectin, cellular matrix material isolated from one or more biological tissues or combinations thereof. One or more cellular growth regions may be formed by painting, tampo printing, transfer printing, screen printing, ink jet deposition, lift off method of lithography, embossing, soft lithography, molding, casting, or etch and fill (damascene). The one or more cellular growth regions may be formed on a surface of a substrate or formed within a cavity in a substrate. Cells to be evaluated include, but are not limited to human cells. A device of the invention may comprise one or more reference regions of fibronectin to which patterns formed by cell growth on the cellular growth regions comprised of matrix materials are compared.

Another embodiment of the invention includes a method for determining the invasive potential of cells comprising: a) depositing the cells to be evaluated on a substrate having one or more cellular growth regions comprised of a matrix material; b) incubating the cells on one or more cellular growth region under conditions that allow for migration, growth, or migration and growth of the cells; c) identifying cellular patterns that arise from the migration, growth, or migration and growth of the cells on one or more cellular growth regions; and d) interpreting the cellular patterns to determine the invasive potential of the cells. In certain embodiments, at least a portion of one or more cellular growth regions are of a thickness sufficient to permit the cells to penetrate, remodel and become embedded in the matrix material. The method may further comprise treating the cells grown on one or more cellular growth regions with: e) a cell permeabilizing agent; f) an endonuclease ALU, a nuclease, e.g., a DNase, or both; and g) a nucleic acid stain. The cells to be evaluated may be a mixture of invasive cells, suspected of being invasive cells and/or non-invasive cells. In certain aspects, the cells to be evaluated may be treated with an MSP I enzyme, which may be followed by exposure to a nucleic acid stain, such as ethidium bromide.

In certain aspects of the invention, at least a portion of one or more regions of matrix material are of a thickness sufficient to permit the cells to penetrate, remodel, and embedded in the matrix material. The method may comprise treating the cells grown on one or more cellular growth regions with: e) a cell permeabilizing agent; f) an endonuclease ALU, a nuclease DNase or both; and g) a nucleic acid stain. In certain aspects, the cells to be evaluated may be treated with an MSP I enzyme, which may be followed by exposure to a nucleic acid stain, such as ethidium bromide.

In another aspect of the invention, the mixture of cells are deposited upon a layer of normal or non-invasive cells grown on the one or more cellular growth regions, as described herein. In various embodiments, at least a portion of one or more cellular growth regions are of a thickness sufficient to permit the invasive cells to penetrate, remodel and become embedded in the matrix material. The method may further comprise treating the mixture of cells with: e) a cell permeabilizing agent; f) an endonuclease ALU, a DNase or both; and g) a nucleic acid stain. In certain aspects, the cells to be evaluated may be treated with an MSP I enzyme, which may be followed by exposure to a nucleic acid stain, such as ethidium bromide.

Embodiments of the invention include a method for determining a tissue or organ site to which invasive cells may metastasize comprising: a) depositing the invasive cells to be evaluated on a substrate having one of more cellular growth regions comprised of a matrix material, wherein the matrix material is obtained or derived from tissues or organs to which the invasive cells may migrate; b) incubating the invasive cells on one or more cellular growth regions under conditions that allow for migration, growth, or migration and growth of the cells; c) identifying cellular patterns that arise from the migration, growth, or migration and growth of the cells on one or more cellular growth regions; and d) interpreting the cellular patterns to determine a tissue or organ site to which invasive cells may metastasize. In certain aspects, at least a portion of the cellular growth region is of a thickness sufficient to permit the cells to penetrate, remodel, and become embedded in the matrix material. The method may further comprise treating the cells with: e) a cell permeabilizing agent; f) ALU endonuclease, DNase or both; and g) a nucleic acid stain. In certain aspects, the cell, tissue or organ to be evaluated may be treated with an MSP I enzyme, which may be followed by exposure to a nucleic acid stain, such as ethidium bromide.

Still further embodiments may include a method for screening a compound, drug or pharmaceutical composition for efficacy as an anti-cancer compound, drug or pharmaceutical composition comprising: a) depositing cancerous or pre-cancerous cells on a substrate having one or more cellular growth regions comprised of a matrix material; b) treating the cancerous or pre-cancerous cells with the compound, drug or pharmaceutical composition to be evaluated; c) incubating the cancerous or pre-cancerous cells on one or more cellular growth regions under conditions that allow for migration, growth, or migration and growth of the cancerous or pre-cancerous cells; d) identifying cellular patterns that arise from the migration, growth, or migration and growth of the cancerous or pre-cancerous cells on one or more cellular growth regions; and e) interpreting the cellular patterns to determine the effects of the compound, drug, or pharmaceutical composition on the cancerous or pre-cancerous cells. In certain aspects, at least a portion of one or more cellular growth regions are of a thickness sufficient to permit the cells to penetrate, remodel, and embed in the matrix material. The methods may include the evaluation of the efficacy of an anti-cancer drug or pharmaceutical composition upon nucleated cells or enucleated cells (cytoplasts). The methods may further comprise treating the cancerous or pre-cancerous cells with: f) a cell permeabilizing agent; g) an ALU endonuclease, a DNase, or both; and h) a nucleic acid stain. In certain aspects, the cells to be evaluated may be treated with an MSP I enzyme, which may be followed by exposure to a nucleic acid stain, such as ethidium bromide.

In yet still further embodiments a method for determining the efficacy of an anti-cancer drug or a pharmaceutical composition comprising: a) depositing cancerous or pre-cancerous cells that have been treated with the anti-cancer drug or pharmaceutical composition to be evaluated on a substrate having cellular growth regions comprised of a cellular matrix material; b) incubating the cells on the cellular growth regions under conditions that allow for migration, growth, or migration and growth of the cells; c) identifying cellular patterns that arise from the migration, growth, or migration and growth of the cells on the cellular growth regions; and d) interpreting the cellular patterns to determine the effects of the anti-cancer drug or pharmaceutical composition on the cells is contemplated. In certain aspects, at least some portion of the cellular matrix material is of a thickness sufficient to permit the cells to penetrate, remodel, and embed in the matrix material. The effects of an anti-cancer drug or pharmaceutical composition upon nucleated cells or enucleated cells (cytoplasts) can be evaluated. The method may further comprise treating the cancerous or pre-cancerous cells with: f) a cell permeabilizing agent; g) an ALU endonuclease, a DNase, or both; and h) a nucleic acid stain. In certain aspects, the cells to be evaluated may be treated with an MSP I enzyme, which may be followed by exposure to a nucleic acid stain, such as ethidium bromide.

Still other embodiments include methods for detecting compounds that initiate, promote or potentiate cancerous behavior in cells comprising: a) depositing normal or non-invasive cells on a substrate having one or more cellular growth regions comprised of a matrix material; and b) treating the cells with the compound to be evaluated; c) incubating the cells on one or more cellular growth regions under conditions that allow for migration, growth, or migration and growth of the cells; d) identifying cellular patterns that arise from the migration, growth, or migration and growth of the cells on the cellular growth regions; and e) interpreting the cellular patterns to determine whether the compound initiates, promotes and/or potentiates cancerous behavior in the cells. In some aspects, at least some portion of one or more cellular growth regions is of a thickness sufficient to permit the cells to penetrate, remodel, and embed in the matrix material. The methods may further comprise treating the cancerous or pre-cancerous cells with: f) a cell permeabilizing agent; g) an ALU endonuclease, a DNase, or both; and h) a nucleic acid stain. In certain aspects, the cells to be evaluated may be treated with an MSP I enzyme, which may be followed by exposure to a nucleic acid stain, such as ethidium bromide.

Embodiments of the invention include methods for detecting compounds that initiate, promote or potentiate cancerous behavior in cells comprising: a) depositing normal or non-invasive cells that have been treated with the compound to be evaluated on a substrate having one or more cellular growth regions comprised of a matrix material; b) incubating the cells on one or more regions of matrix material under conditions that allow for the migration, growth, or migration and growth of the cells; c) identifying the cellular patterns that arise from the migration, growth, or migration and growth of the cells on one or more cellular growth regions; and d) interpreting the cellular patterns to determine whether the compound initiates, promotes or potentiates cancerous behavior in the cells. In certain aspects, at least some portion of one or more cellular growth regions are of a thickness sufficient to permit the cells to penetrate, remodel and embed in the matrix material. The efficacy of an anti-cancer drug or pharmaceutical composition upon nucleated cells or enucleated cells (cytoplasts) may be evaluated. The methods may further comprise treating the cancerous or pre-cancerous cells with: a cell permeabilizing agent; an ALU endonuclease, a DNase, or both; and a nucleic acid stain. In certain aspects, the cells to be evaluated may be treated with an MSP I enzyme, which may be followed by exposure to a nucleic acid stain, such as ethidium bromide.

In further embodiments of the invention, the devices and methods of the invention may be employed in the in vitro assessment of the efficacy of therapeutic agents against refractory invasive cancers. It is convenient to juxtapose layers of thin and thick matrix in such devices such that the cells growing on thin matrix serve as procedural controls for the cells growing on thick matrix. Cancer cells from a patient or other source are grown on thin and thick layers of extracellular matrix to the point that tumor nests are formed by the cells on the thick matrix. The cells are then exposed to a therapeutic agent, typically by addition of the agent to the cell growth medium, and the results of such exposure observed. An effective agent will label all of the cells grown on thin matrix and will penetrate into the clusters of cells forming the tumor nests on thick matrix. Efficacy correlates with increasing penetration of agent. Further evidence of efficacy may be obtained by observation of tumor nests for signs of necrosis and/or apoptosis upon prolonged exposure to the therapeutic agent. Additional indications of efficacy may be obtained by exposing the cells treated with the therapeutic agent to nucleases such as ALU or DNAase in the manner described herein.

In still further embodiments of the invention, cells are grown on thin and thick matrix, or gradients thereof, using devices and methods of the invention. The cells grown on thin and thick matrix are harvested separately. Harvesting is preferably performed mechanically in order to avoid artifacts that may accompany the use of chemical methods such as treatment with chelating agents such as EDTA or EGTA or with proteolytic enzymes such as trypsin that are commonly employed for this purpose. The cells harvested from thin and thick matrix are then separately prepared for application to “gene array” chips such as an Affymetrix II Microarray (Affymetrix). The data obtained from these chips is then analyzed, preferably using a paired T-test, correlation analyses or similar methods, to identify the specific genes for which expression differs between cells grown on thin and thick matrix. Methods of the invention include the identification of a therapy or therapeutic target for modulating the phenotype of a cell. The phenotype may be a senescent or invasive phenotype. The phenotype desired is based on the therapeutic effect desired, for example an invasive phenotype may be more sensitive to a particular therapy, whereas a senescent phenotype may be resistant to the therapy, but the probability of metastasis is decreased. Minimizing metastasis may be used in conjunction with surgery or other cancer therapies.

Embodiments of the invention include additional methods for use in analysis of standardized panels of tumor tissues and/or cells against which the efficacy of potential anti-cancer agents can be assessed. Some such panels are comprised of living tissues excised from patient tumors while others are comprised of cultured tumor cells grown on a substrate or in suspension. Improved tumor panels can be constructed by growing tumor cells of the requisite type or types using devices and/methods of the present invention. In certain aspects, the tumor panels are more representative of the environments in which in vivo tumor growth occurs and therefore provide for more realistic assessment of the efficacy of anti-cancer agents. These improved panels provide a more defined and controlled environment than do panels comprised of tumor tissues and therefore facilitate comparative assessments. Invasive tumor cells grown on thin matrix are typically more susceptible to the action of anti-cancer agents than are the same cells grown on thick matrix. Thus the apparent efficacy of agents tested against invasive cells grown on thin matrix will be artifactually elevated. Conversely, the invasive behavior of tumors grown on thick matrix can be less than that of the same cells grown on thin matrix. This can mask the efficacy of the agent being tested. More accurate testing can be performed if cells grown on both thin and thick matrix are employed as reference materials in the manner embodied in this invention.

In further embodiments, methods include the direct utilization of specimen cells in a fluid suspension rather than requiring that the cells to first be transferred to a solid substrate. The methods include culturing cells that may or may not be derived from patient specimens. If cells are grown in monolayer culture or are included in a biopsy or other tumor sample, the cells may be mechanically harvested. Cells are pelleted by centrifugation. The cellular pellet is re-suspended in an appropriate buffer; incubated for an appropriate time at room temperature; spun down; and resuspended. Propidium iodide is added to an aliquot of this suspension. Alu I restriction enzyme is added to the remaining cell suspension and the preparation incubated at 37° C. Aliquots of this mixture are taken for evaluation at various times, for example 0 (baseline), 1, 3, and 5 hours after the addition of ALU. Propidium iodide is added to each of the digested samples. The resulting digested and stained cell suspensions are analyzed according to standard methods using FACS analysis or a similar analytical method. An aliquot of cell suspension may be treated with PI after permeabilization, but not digested with ALU serves as a reference for the amount of DNA present in each of the cell preparations prior to the start of treatment.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. FIG. 1A shows highly invasive MUM-2B cells growing on extracellular matrix protein. The thickness of the matrix protein varies from approximately 35 nm (absorbed protein) on the left (arrowhead mark) to approximately 1 mm on the right (long arrow mark). On matrix thicknesses of up to about 50 microns (to the left of the black line) the cells form random monolayer aggregates. In the matrix thickness range between about 50 and 150 microns (check mark), the cells form cordlike structures that mimic tumor vascularization. At thicknesses above about 150-250 microns, the cells form cylindrical or spheroidal tumor nests that are surrounded by remodeled matrix protein. FIG. 1B shows non-invasive OCM-1a cells growing on a similar matrix gradient (thickness increases to the right). These cells form discrete random aggregates at all thicknesses of matrix protein. FIG. 1C shows non-aggressive cells forming spheroids on any thickness of matrix.

FIGS. 2A-2D. FIG. 2A shows a bright field image of laminin matrix protein deposited at about 100 microns thickness in the shape of the numeral “3” on a uniform layer of absorbed laminin protein about 35 nm thick. MFC-10A breast cancer cells were deposited uniformly over this entire area and digested with DNAase. FIG. 2B shows a fluorescence image of this same area after staining with the DNA stain ethidium bromide. The localization of the cellular DNA to the region of thickest matrix protein is evident. FIGS. 2C and 2D show images of the central portion of the region shown in FIG. 2B at progressively higher magnifications. The cells growing on the thin matrix were unaffected by the action of DNAase. The small fluorescent objects in the area of absorbed matrix protein surrounding the cells are nucleoli that remained after the DNA in the cells was digested by the DNAase treatment.

FIG. 3. The drawing shows a “wedge” or “gradient” version of the chip consisting of lines of matrix material having thicknesses ranging from about 35 nm (the thickness of the film formed by absorbing proteins onto glass from solution) to about 1 mm.

FIGS. 4A-4E. FIG. 4A shows a portion of a chip that is similar to that in FIG. 3 except that the lines of matrix are arranged in a grid pattern. The lines are fibronectin or collagen and the squares are laminin. FIG. 4B and 4C show aggressive MB231 cells on this chip and FIG. 4D and 4E show non-aggressive MCF10a cells on the same chip. Both sets of cells have been treated with ALU for 60 minutes. The top image in each pair is in phase contrast while the bottom image pair is in fluorescence after staining with ethidium bromide. Aggressive cells on thick (about 1 mm) laminin remain intact while the aggressive cells on fibronectin or collagen and non-aggressive cells on both matrix proteins are heavily degraded.

FIGS. 5A-5H. FIG. 5A, 5C, 5E, and 5G show the cells imaged in phase contrast while FIG. 5B, 5D, 5F, and 5H show the same cells imaged in fluorescence after ethidium bromide staining. The FIG. 5A, 5B, 5E, and 5F are controls showing the cells after 30 and 150 minutes of digestion, respectively. FIG. 5C, 5D, 5G, and 5H show cells that have been treated for about 15 minutes with an experimental polyamine anti-cancer drug before being digested under the same conditions as the corresponding controls. In both cases, the polyamine stabilizes the DNA against degradation.

FIGS. 6A-6C. Studies showing the sensitivities of fibroblasts (FIG. 6A), OCM 1 a (FIG. 6B)(poorly invasive melanomas), and MUM 2B (FIG. 6C)(highly invasive melanomas) after 24 hours of incubation with MSP I. Note that fibroblast nuclei are completely digested in 24 hours. OCM 1a nuclei showed some focal residual staining, while MUM 2B nuclei exhibited complete stability and sequestration from the methylation-specific enzyme.

FIGS. 7A-7B FIG. 7A shows flow cytometer fluorescence intensity histogram plots measured for each of WI-38 fibroblasts (normal cells); OCM1 (a poorly invasive a primary uveal melanoma); M619 (a highly invasive primary uveal melanoma; and MUM2B (a highly invasive metastatic uveal melanoma) at 1, 3 and, 5 hours exposure to Alu I restriction enzyme followed by staining with PI. FIG. 7B shows a composite of fluorescence intensity for each time point.

### DESCRIPTION OF ILLUSTRATIVE EMBODIMENTS

Embodiments of the invention address various limitations of current diagnostic devices, compositions, and methods, as described herein. The invention relates to compositions, methods, and/or devices for detecting and/or determining the invasiveness of cells and for differentiating between degrees of cellular invasiveness. In certain embodiments, the invention relates to devices and methods for evaluating the interactions between cells, and between cells and matrix materials wherein the cellular distribution patterns formed as a result of such interactions are indicators of the invasive potential(s) of the cells or one or more cells of a cell mixture, tissue, organ or other biological sample. Furthermore, such devices and methods can provide indications of the preferred sites of metastasis of invasive cells; the efficacy of an anti-cancer drug applied to such cells; and the potential for agents to promote or enhance tumor growth or metastasis.

In contrast to the present invention, current methods of cell culture used in the investigation of cancerous cells are based upon cell growth in suspension or, not withstanding hyper-confluent growth, as a monolayer on a solid support. When performed in conformance with accepted practices, suspension cultures largely permit only the attachment of cells to other cells via cell-cell interactions. Under these conditions, normal cells or cancer cells of low invasive potential tend to form suspensions consisting of isolated cells while highly aggressive cancer cells cultured under similar conditions can form cellular aggregates of significant size (millimeters in diameter).

Monolayer cell cultures are permissive of both cell-cell and cell-substrate interactions. In order to encourage monolayer cell growth, accepted practice is to absorb a layer of serum proteins that is on the order of a few hundred nanometers in thickness onto the solid support. This protein layer facilitates adhesion of the cells to the solid support while, by virtue of having a thickness that is only a small fraction of that of a cell, constraining the adhered cells to a single plane. Under these conditions, the cell growth pattern observed tends to be characteristic of the type of cell being grown. An alternative, but less commonly employed approach is illustrated by the “In-Vitro Angiogenesis Assay Kit” marketed by Chemicon International (Temecula, Calif.). In this approach, the cells are grown on a relatively thick (typically in excess of 1 mm) layer of a gelatinous cell culture medium in a manner that is analogous to that for microbial cell cultures. Some cell types such as those used in the referenced angiogenesis assay grow on the surface of the gel while others can penetrate and grow within the gel. Again, the growth patterns observed tend to be a function of the cell type.

It is known that cells interact with each other and with the extracellular matrix through various families of cell surface proteins such as the “integrin receptors.” It is further known that cells can generate mechanical tension in their cytoskeletons and that this tension can result in tractional forces being exerted between the cell and the extracellular matrix. Modulation of these forces can cause a cell to change shape and to switch between growth and differentiation. This switching action implies the presence of a relationship between the mechanical interactions of the cell surface and extracellular matrix, as well as intracellular functions such as gene expression and cell cycle progression. One observation that bears upon the present invention is that there appears to be a significant mechanical component to this relationship in that mechanical forces artificially applied to cell surface integrin receptors result in immediate changes in nuclear and cytoplasmic cell morphology that are identical to those seen under biological conditions.

A second observation that bears upon the present invention is that the morphologies of cells and assemblies thereof are strongly influenced, if not controlled by the nature and thickness of the extracellular matrix with which the cell is in contact. Ancillary to this are the observations that this influence is modulated by whether or not the cell is cancerous; by the degree of invasiveness of the cell; and by other cells that may be in contact with the cell in question. It has further been observed that many of these same morphological changes are recapitulated by enucleated cells (cytoplasts).

In the present invention, it has been determined that growing cells on layers of extracellular matrix that are intermediate in thickness between those indicated above results in cell growth patterns that are relatively independent of the cell type, but that are strongly dependent upon the invasive potential of the cell. For this purpose, three distinct thickness regimes can be defined. Layers of serum proteins, which include, but are not limited to the extracellular matrix proteins collagen and laminin, absorbed on a solid support are typically less than a few hundred nanometers in thickness. Cells grown on such layers appear not to penetrate this layer nor do they appear to remodel the extracellular matrix proteins found therein. Cells grown on layers of extracellular matrix that are greater than a 50 microns, but less than 100 microns in thickness (thin matrix) can penetrate and remodel the layer of matrix protein, but do not become embedded in it. At layer thicknesses greater than about 100 microns, cells can penetrate, remodel and become embedded in the matrix protein.

With the specific exception of normal vasculogenic endothelial cells, normal cells and cancer cells of low invasive potential form clusters containing small numbers of cells when grown to less than confluent density on serum proteins absorbed to a solid support or on layers of extracellular matrix ranging from less than 50 microns to over 1000 microns in thickness. These cells do not show discernable patterns of cell distribution even when grown in the presence of saturating concentrations of cell growth factors or under hypoxic conditions. Normal vasculogenic epithelial cells grown to confluent density on absorbed serum proteins form characteristic “cobblestone” monolayers. These normal endothelial cells form distinctive cord-like structures when grown on thin matrix and networks of these cords when grown on thick matrix. The formation of these vasculogenic cords, which are known to be precursors to the formation of blood vessels, is prevented by the addition of anti-angiogenic agents such as the drug TNP-470 (a derivative of flimagillin) to the culture medium.

In a similar manner, highly aggressive cancer cells also form aggregates having no discemable pattern when grown to sub-confluent density on absorbed serum proteins. Such aggregates, however, tend to be larger than those observed when normal cells and/or non-aggressive cancer cells are grown under the same conditions. When grown on thin matrix, aggressive cancer cells form cord-like structures that are similar in appearance to those formed by normal endothelial cells under comparable conditions. These cords consist of groups of cancer cells surrounded by remodeled matrix protein and can conduct fluids for short distances. They are not, however, vasculogenic as their formation is not inhibited by the addition of anti-angiogenic agents to the culture medium and there is no evidence that they can mature into blood vessels. Such cords are described as exhibiting “vasculogenic mimicry.” On thick matrix, aggressive cancer cells form spheroidal to cylindrical tumor “nests” consisting of tumor cells embedded in and enclosed by complex networks of highly remodeled matrix protein. When viewed perpendicular to the plane of the matrix layer, these nests appear as clusters of cells bordered by distinctive “looping” patterns comprised of an amalgam of tumor cells and extracellular matrix. Similar looping patterns have been observed in tumor biopsy specimens and have been correlated with the presence of highly invasive and metastatic cancers.

The patterns formed by cancer cells are largely independent of cell type except to the extent that the cell types differ in invasive potential. Within the limits imposed by the requirements for cell growth, these patterns are also independent of various cell growth factors, oxygen tension, and similar factors relevant to the cell culture procedures. The patterns produced by normal and non-aggressive cancer cells are not affected by soluble factors produced by aggressive cancer cells. Within certain limits discussed below, each sub-population within a mixed culture of normal, non-aggressive and/or aggressive cells grows and forms patterns independently of the other cell types in the mixture. Within the context of this invention, it has been determined that of all matrix proteins evaluated, only fibronectin and highly denatured type I collagen do not support the formation of the growth patterns that are characteristic of invasive cells and do not protect the chromatin in cells grown on these matrices from digestion by nucleases such as DNase. This permits the use of these matrix materials as “negative” controls, particularly in applications such as drug evaluations where multiple types of matrix materials are employed. Matrix materials may include, but are not limited to collagen, fibronectin, laminin, hyaluronic acid, heparan sulfate, chondroitin sulfate, dermatan sulfate, sulfated proteoglycans, fibrin, elastin, tenascin or combinations thereof. Various materials for making a matrix are known in the art and may be used in conjunction with the present invention.

Strong clinical correlations have been established between the propensity of cancers to form looping patterns on thick matrix or in tissue, and a negative prognosis or outcome due to tumor metastasis. In this context, it is relevant to note that the ability of an aggressive cancer to form such looping patterns depends upon types of both the cancer and the extracellular matrix. Metastatic phenotypes of melanoma, for example, readily form looping patterns in thick layers of collagen, a major constituent of the extracellular matrix at the typical sites of primary tumor formation, and in thick layers of extracellular matrix derived from liver, a preferred site for the metastasis of melanomas, but less readily in thick layers of extracellular matrix derived from bone which is a secondary site of melanoma metastasis. Similarly, metastatic phenotypes of prostate cancer, which initially metastasize to bone, readily form looping patterns in this type of matrix. Conversely, breast cancers which generally do not metastasize to collagen-rich sites do not form looping patterns in thick collagen layers. In other words, the types of matrix that support formation of looping patterns are suggestive of the target sites for metastatic cancers. It is of relevance that, to date, it has not been possible to induce any type of cancer to form looping patterns on thick fibronectin matrix. The clinical significance of this observation is yet to be determined, but it appears that fibronectin can serve as a negative control for assays based upon the formation of cellular patterns on extracellular matrix.

A related aspect of the present invention can be demonstrated when mixtures of normal, minimally invasive, and highly invasive cells are grown together on the same thin or thick matrix. Specifically, normal and minimally invasive cells can coexist in contact for extended periods of time whereas highly invasive cells are highly cytotoxic and cytolytic to normal and minimally invasive cells, and kill such cells within one hour of contact under typical cell culture conditions. This behavior is exhibited only under conditions of cell-cell contact. In other words, highly invasive cells lyse and kill normal and minimally invasive cells that they come into contact with, but have no apparent effect on such cells that are not in physical contact. For example, a dispersion of cells obtained from a tumor specimen and applied on top of a layer of normal cells growing on thin matrix will penetrate and lyse the layer of normal cells in those locations where aggressive cells from the tumor are present, but not where normal and/or non-invasive tumor cells are present. These effects of cell to cell contact can be detected and evaluated through the use of the present invention.

The present invention can be employed to screen compounds, e.g., anti-cancer drugs, for efficacy or, conversely, to screen agents for their ability to promote or enhance the aggressiveness of cancers. In the former case, treatment of aggressive cancer cells with the compound or drug to be evaluated prior to culturing the cells on thick matrix and determining whether the drug treatment suppressed formation of looping patterns. Conversely, agents suspected of promoting or enhancing cellular aggressiveness can be evaluated by treating normal or minimally invasive cells on a matrix with the agent and observing changes in the resulting cellular patterns. In a similar manner, the present invention can be used to determine whether the action of an anti-cancer drug or suspect agent is exerted at the nuclear and/or cytoplasmic level by comparing the patterns produced by treatment of the appropriate cell type with the drug or agent under test with those produced by treatment of cytoplasts or enucleated cells prepared from the same cell type with the same drug or agent.

Another aspect of the present invention is that neither a cell nucleus nor gene transcription is required in order to differentiate between normal, minimally invasive and highly invasive cells on the basis of the morphological patterns formed on various thicknesses of extracellular matrix. In particular, cytoplasts produced by the enucleation of normal and minimally invasive cell types form isolated clusters of cytoplasts when seeded on layers of extracellular matrix while the cytoplasts derived from aggressive cells form cord-like structures that closely resembled those formed by intact aggressive cells on thin matrix. Enucleated highly invasive cells have not, however, been observed to form looping patterns on thick matrix.

Pending patent application Ser. No. 10/862,235, filed Jun. 7, 2004, entitled Quantitative Chromosome Stability Assay, which is incorporated herein by reference in its entirety, describes a method for differentiating between normal and cancerous cells based upon the differences in the susceptibility of chromatin to digestion by an endonuclease, a nuclease, and/or in combination with a protease. In particular, the nuclease DNase preferentially degrades the chromatin in permeabilized normal and non-invasive cells while leaving the chromatin in invasive cells largely intact. The cells used in the assay can be grown in suspension cultures or as monolayer cultures on a solid support coated with absorbed serum proteins.

The susceptibility of cellular chromatin to DNase digestion is further reduced in cells that are grown on extracellular matrix relative to those grown under otherwise identical conditions on serum proteins absorbed to a substrate or in suspension. In the present invention, this enhanced differential susceptibility can be employed to discriminate between normal and invasive cells on and off a matrix. For example, the treatment of Triton X-100 permeabilized cells with DNase for one hour degrades the chromatin in normal and invasive cells on serum proteins and normal cells on matrix, but leaves the chromatin of invasive cells grown on a matrix intact. Adjustment of the conditions such as DNase concentration and duration of exposure permits finer differentiations such as between moderately invasive cells that form degradation resistant cord structures on matrix and the even more degradation resistant highly invasive cells that form tumor nests and looping patterns. This feature, particularly in conjunction with the subsequent treatment of the permeabilized, DNase digested cells with a nucleic acid stain such as, but not limited to ethidium bromide, doxorubicin or bisbenzamide provides a significant enhancement in the signal to noise ratio when capturing and evaluating the cellular patterns produced by this invention.

The observed differences in chromatin stability toward digestion by nucleases, endonucleases, and/or in combination with proteases between normal/non-invasive and invasive cells and between cells grown on serum proteins versus those grown on matrix strongly suggest that significant differences in chromatin sequestration and gene expression exist between these cell types and growth conditions. Another indicator that points to this same conclusion is based upon the evaluation of gene expression using “gene array chips.” Comparing gene expression between the two phenotypes revealed 1081 differences with 546 genes being up-regulated and 535 genes being down regulated in M-619 cells grown on matrix relative to the same cells grown on absorbed serum proteins. Other changes in expression pattern can be shown between normal, minimally invasive, and invasive cells; between cells grown on layers of different matrix proteins; and between isolated cells and cells that are in contact with other cells.

Pending patent application Ser. No. 10/862,235, filed Jun. 7, 2004 entitled Quantitative Chromosome Stability Assay, which is incorporated herein by reference, discloses a method that may be used in conjunction with the present invention. The Quantitative chromosome stability assay is based upon the susceptibility of chromatin to digestion by certain endonucleases, nucleases, and proteases, which reflect the degree of invasiveness of the cell containing or providing the chromatin.

The inventors hypothesize that methylation may generally increase at the level of higher order chromatin structure throughout the genomes of more invasive cells. Typically, methylation of specific genes using MSP PCR is detected with a range of molecular “kits” available from a variety of companies, for example, Serologicals Corporation (Norcross, Ga.), OncoMethylome Sciences S.A. (Durham, N.C.), and others. Qiagen (Valencia, Calif.), for example, has developed MSP PCR to employ methylation-specific PCR (MSP) for several specific promoters. Methylation-specific PCR (MSP) of these promoters, it is claimed, allows precise mapping of DNA methylation patterns in GC-rich regions of DNA. It is assumed that hypermethylation of promoter regions is often a decisive factor in inactivation of tumor suppressor genes in human cancers.

However, most tumor suppressor genes identified to date have been identified only in familial cancers where linkage studies were possible. The incidence of these cancers is approximately 1% of all cancers according to the NCI's “cancer incidence list.” This means that 99% of cancers have no marker genes by which to detect methylation, which is the principal reason-why the MSP PCR industry, researchers, and experts are in agreement that the challenge ahead is to identify the important genes in the major sporadic cancers where a linkage analysis is not possible, e.g., sporadic breast, prostate, bladder, lung, and most other cancers. Even among the familial cancers, where tumor suppressor genes have been identified, most authors refer to the target gene being tested as “putative tumor suppressor genes.”

Because of these difficulties, an assay has been developed by the inventors that employs chromatin testing of populations of cells under normal physiological ionic conditions in lysed cell models, in assays that employ flow cytometry, and in smear preps similar to Pap smears. The test is based upon the cell as an integrated mechanical unit whose genetic sequestration and exposure is controlled not only from the level of histone octamers or topoisomerases (Maniotis et al., 1997; Bojanowski et al., 1998), but at the level of higher order chromatin structure (Karavitis et al., 2003; Maniotis et al., 2003). By testing the sensitivity of Alu, Eco RI, Mbo, Hind-1, PST-1, and other specific and non-specific nucleases and proteases, the inventors have determined that disulfide-rich proteins differentially sequester Alu sequences as cells increase their invasive behavior and as cells become associated with specific matrix molecules and matrix thickness. Furthermore, cells differentially sequester Alu sequences depending on their cytoskeleton organization.

MSP I digestion sensitivity or digestion insensitivity was tested as a generalized property of nuclei within cells of increasing invasive and malignant behavior. The results of these studies, described below, show that sequestration and exposure of methylated sites occurs at the level of higher order chromatin folding, and not only at the level of specific putative cancer genes or gene sequences.

I. Matrix Materials

Certain extracellular matrix materials such as collagen, laminin and fibronectin are available in various types, forms, grades, purities and modes of preparation from commercial sources and can be employed in the practice of the present invention. Other extracellular matrix materials can be prepared by methods known in the art, for general exemplary methods see U.S. Pat. Nos. 6,372,494, 5,830,708, and 5,162,114 each of which is incorporated herein by reference. Matrix materials may include proteinaceous (e.g., extracellular matrix materials), and non-proteinaceous materials, or combinations thereof. Matrix materials may include, but are not limited to collagen, fibronectin, laminin, hyaluronic acid, heparan sulfate, chondroitins, chondroitin sulfate, dermatan sulfate, sulfated proteoglycans, fibrin, elastin, tenascin, actins, cadherins, ICAMs/VCAMs, integrins, kinesins, merosins, microtubule-associated proteins, myosins, neurofilaments, profilactin, profilins, pronectin, selectin, thrombospondins, troponins, tubulin, vimentin, vitronectin or combinations thereof. Matrix material refers to the a matrix material that provides at least physical support to a cell deposited thereon and may include physiological support for growth and other life processes. Extracellular matrix from liver tissue may, by way of example, be prepared as follows. Fresh liver tissue is washed with ice cold deionized or distilled water; minced into small (e.g., 1 mm cube) fragments; and suspended and homogenized in 0.02 M phosphate buffered saline (PBS) pH=7.3 (0.5 mM NaH₂PO₄, 1.9 mM Na₂HPO₄, 17.9 mM NaCl). The resulting suspension is allowed to stand for 5 min at 4° C. before being centrifuged at 2500×G for 15 min. The supeemate containing liver extracellular matrix protein is recovered by filtration through a membrane filter having a 0.22 micron pore size and stored at −4° C. Extracellular matrix proteins from other tissues such as, but not limited to lymph node, thymic husk, bone, brain, and lung can be isolated using analogous methods and/or corresponding methods known in the art.

II. Device Fabrication

A device suitable for the practice of this invention may consist of one or more regions or zones of material of defined composition and thickness that is deposited or formed upon or within a substrate. In certain embodiments, such devices include multiple regions or zones of material deposited or formed upon or within a substrate. Each region may have the same or different composition and/or thickness as one or more other regions of material on the same substrate. In some instances it is desirable for the region(s) to abut one another. In other instances it is desirable for the regions to be spaced apart or divided by a barrier.

Glass microscope coverslips, which have the benefits of being flat, smooth, uniform in thickness, transparent, and relatively inert as well as having a surface chemistry that can readily bind and immobilize proteins and cells, comprise one convenient substrate for the preparation of the devices of the present invention. Silicon wafers and especially silicon wafers having a grown or deposited oxide, nitride or other coating of appropriate thickness is another suitable substrate material, albeit one that has the disadvantages of being opaque and highly reflective in visible light. Similarly, polymeric materials including, but not limited to virgin poly-styrene that has been formulated with no fillers, plasticizers or other additives and which has been processed under conditions that minimize the amount of unreacted monomer and low molecular weight oligomers in the polymer can be used for this purpose. The surfaces of such substrates can further be coated, conditioned, modified or otherwise prepared to improve protein adhesion and other attributes. In various embodiments, glass surfaces may be freed of organic contaminants through the use of a formulation such as 3 parts of 30% H₂O₂ combined with 7 parts of concentrated H₂SO₄. The surface may then be further processed to remove metallic contaminants and to place the glass surface in a hydrophobic or hydrophyllic state by treatments with formulations such as 3 parts of 30% H₂O₂ plus 7 parts of either concentrated HCl or concentrated NH₄OH. Such surfaces can be further modified by treatment with a silane reagent such as chloro-dimethyl-aminopropyl silane. Polymeric surfaces may be similarly prepared by means such as gas plasma or corona discharge treatments and the like. The substrate materials and treatment methods cited are meant to be exemplary and do not comprise an exhaustive or comprehensive list. Numerous other such materials and methods are known to those skilled in the art and may be employed in the practice of this invention without departing from the spirit of the invention.

In like manner, numerous methods exist and are known to those skilled in the art for the deposition or formation of regions or zones of matrix proteins on or embedded in a selected substrate. As noted above, layers of proteins including matrix proteins that are a few hundred nanometers in thickness can be absorbed onto the substrate surface from a solution of the protein(s). Such absorbed layers are useful in their own right as substrates upon which cells can grow and as layers that facilitate the adhesion of other subsequently deposited or formed protein layers. The entire surface of a substrate can be coated with a uniform protein layer of controlled thickness by a process such as dipping, spin coating or spray coating, all of which are well known to those skilled in the art. The thickness of the protein layer formed via such processes is a sensitive function of numerous process variables such as, but not limited to the protein concentration, surface tension and viscosity of the protein solution; the surface free energy of the substrate; withdrawal rate or spin speed profile; ambient temperature and humidity; air flow velocity and distribution; and the like. The thickness of the layer formed can be controlled by adjusting these and similar parameters and/or by applying multiple layers of the same or different proteins to the substrate.

Writing, painting, and printing processes, including, but not limited to screen and “ink jet” printing, represent another class of means for the deposition of zones of matrix proteins upon or within a substrate. As an example of this class, zones of matrix that are approximately 10 microns wide by 100 microns long by 20-50 microns thick can be “written” on the substrate using a “pen” prepared by drawing a fine glass rod down to a point that is approximately 5 microns in diameter using a microforge. This pen can be mounted in a micromanipulator, dipped in a solution of matrix proteins, and the protein solution adhering to the pen transferred to the substrate by bringing the pen tip into contact with the substrate and moving the pen tip relative to the substrate in a manner such as to inscribe the protein solution on the substrate in the desired pattern. Alternatively, a droplet of matrix protein solution of the desired volume can be placed on the substrate and the pen used to spread this solution over the desired region of the substrate.

Another example of such a process utilizes a pad of a compliant material such as silicone rubber that has been formed into a negative image of the desired deposition pattern by any of several methods such as casting against a microfabricated tool that are known to those skilled in the art. The figured surface of this pad can be dipped into a solution of the desired matrix protein and the protein solution adhering to the pad can be transferred to the substrate by bringing the figured face of the pad into contact with the substrate. In most cases, a suitable “ink” for such processes can be prepared by dissolving the desired matrix protein(s) in a solvent consisting of 0.15 M NaCl and 0.002 M MgCl₂ in deionized water at a concentration range, including all intervening values, of about 0.5, 1, 2, 3, 4, 5, 6, 7, 8, or 9,to about 11, 12, 13, 14, 15, 16, 17, 18, 19 or 20 mM with about 10 mM being the preferred concentration for most matrix materials or proteins. For example, the ranges of 0.5 mM to 20 mM, 9 mM to 20 mM, 9 mM to 11 mM, and 0.5 mM to 11 mM are included. The thickness of the protein layer formed via such a process is a sensitive function of numerous process variables such as, but not limited to the protein concentration, surface tension and viscosity of the protein solution; the surface free energy of the substrate and the pen tip or pad; contact pressure; rate of inscribing; ambient temperature and humidity; air flow velocity and distribution; and the like. The thickness of the layer formed can be controlled by adjusting these and similar parameters and/or by applying multiple layers of the same or different proteins to the substrate.

Yet another class of means for preparing zones of matrix proteins on or within a substrate are based upon the well known damascene or “etch and fill” process. Such processes create a negative relief image of the pattern to be formed on or in the surface of the substrate. This negative relief image is then filled with matrix protein. In one of the many known variants of such a process, a mask is created in a layer of a compliant material such as a silicone rubber by a method such as casting the rubber on a suitable tool bearing the negative relief image of the desired pattern or by cutting or punching holes in a sheet of silicone rubber in accordance with the desired matrix deposition pattern. Entrapment of the matrix protein in a supporting material such as an open cell foam also falls within this category.

An alternative means of forming such a mask is to apply a layer of photoresist of a desired thickness to the substrate and create the desired negative relief pattern in the photoresist by standard photolithography techniques. The term photoresist generally refers to a viscous polymer resin (solution) containing some photochemically active polymer (PAC), which is typically rendered insoluble or soluble, relative to a wash solution, by exposure to light. By means of a photoresist, a selected pattern can be imaged on a substrate. Areas of a positive photoresist not exposed to electromagnetic radiation may be removed by a washing process. Either a liquid resist such as is used in semiconductor manufacture or a film resist such is used in the manufacture of printed circuit boards may be used for this purpose. Compositions of photosensitized gelatin such as are used in the preparation of volume and phase holograms may similarly be used for this purpose although potassium dichromate, the preferred photosensitizer in most such compositions, can be cytotoxic and must be completely removed from the resulting mask before use. Positive acting resists are most convenient in this application as they facilitate building multi-layer structures in which different zones of matrix on the substrate have different thicknesses. Negative acting resists can, however, likewise be used but with some restrictions on the locations of relief areas in the various layers or with the necessity of ancillary process steps such as hexamethyldisilazane (HMDS) or deposited intermediate etch stops. In any case, a suitable photoresist material applied, photopattemed, developed, and hardened in accordance with the procedures specific to the particular resist material can form a suitable mask for the deposition of matrix protein patterns on a substrate.

Yet another means of preparing a substrate for this process is to cast, mold, emboss, engrave, etch, or hot stamp a suitable relief pattern into the surface of the substrate using methods that are well known to those skilled in the art. Such means are closest to the historical damascene process and offer the benefit that the exposed surfaces of the matrix protein features created in such substrates lie in the plane of the surface of the substrate. The presence of potentially deleterious materials is also avoided.

A substrate prepared by any of the methods described above can be used as a mold in which matrix protein(s) is/are cast. In such instances where the entrance face of the mask has features that lie in different planes, the cavities in the mask can be filled by dispensing a volume of matrix protein solution that has been pre-calculated to appropriately fill the cavity. This same method can be used to fill the cavities in masks where the entrance face of the mask lies in a single plane. In this case, the fluid volume dispensed can be sufficient to completely or partially fill the cavity. Partially filling cavities allows zones of matrix of different thicknesses to be created using a mask having a single thickness. As an alternative, the entire surface of the mask can be flooded with matrix protein solution thus filling all of the cavities and the excess solution removed by wiping with a doctor blade, spinning, decantation or similar method.

Yet another method for the formation of patterned matrix proteins on a substrate is described in U.S. patent application 60/427,646, filed Nov. 19, 2003 entitled “Three Dimensional Multilayer Microstructure of Cells and Biopolymers Created by Microfluidic Layer-by-Layer Technique”.

Some matrix proteins form stable gels that are directly suitable for use in the present invention upon being cast or applied to a substrate. Many matrix proteins do not form adequately stable gels in the as deposited or as cast condition and may, for example, dissolve when exposed to cell culture conditions. For this reason, in some instances it may be desirable to bake the deposited or cast matrix protein at 55° C. for approximately one hour to denature the protein and render it both insoluble and mechanically durable. After denaturing of the matrix protein, silicone rubber and similar masks may be removed from the substrate to leave isolated islands of matrix protein separated by bare or serum protein coated substrate. Photoresist masks, particularly those formed using negative acting photoresists, are difficult to remove from the substrate without damaging the zones of matrix protein. Such masks are best left in place during use of the device. Some positive acting photoresists, even after hard baking, can be removed from the substrate without damaging the matrix proteins if the photoresist to be removed is suitably exposed to high intensity UV light to render it soluble in a suitable solvent.

III. Cell Preparation from Tissues

Cells can be isolated from tissues such as tumor biopsies by methods known to those skilled in the art (for a general review see Freshney, 1987). Such methods are generally similar to those described for the isolation of extracellular matrix protein from liver except that the tissue may be incubated with or homogenized in a medium that contains proteolytic enzymes such as trypsin to disrupt cell-cell interactions and that the desired cells are found in the cellular pellet rather than the supernatant.

Cell culture can be performed in accordance with methods known to those skilled in the art. In most instances, a suitable growth medium consists of DMEM (BioWhittaker, Walkersville, Md.) supplemented with 10% fetal calf serum and, where relevant, suitable concentrations of cell growth factors such as, but not limited to basic fibroblast growth factor, transforming growth factor β, vascular epithelial growth factors, interleukins and other such agents as may be required for the proper growth of the particular cell type(s) being cultured. In certain embodiments antibacterial or antifungal agents are not used in the culturing of cells for use in the practice of this invention as such agents are known to interfere with the differentiation potential of primary cell types. Cell culture is performed at 37° C. under an atmosphere consisting of approximately 5% CO₂/balance air.

IV. Data Capture & Interpretation

Growth and pattern formation by cells can be monitored visually and/or can be captured as electronic images for subsequent quantitative analysis by means of microscopic imaging that are well known to those skilled in the art, for general methods see Current Protocols in Cell Biology (2001); Murphy, Fundamentals of Light Microscopy and Electronic Imaging (2001). One suitable microscopy platform for visual imaging and electronic image capture consists-of a Leica DM IRB inverted microscope (Leica, Wetzlar, Germany) equipped for transmitted light, phase contrast, differential interference contrast and epi-fluorescence visual and electronic imaging at magnifications of 20×, 40×, and 63×. This microscopy platform may also equipped with means to maintain the specimen being imaged at any desired temperature, most commonly 25° C. or 37° C. to facilitate the monitoring of the time courses of the reactions over extended periods of time. A comparably equipped upright microscope such as a Leica model LS or LB may also be employed.

Images of the specimens can be captured electronically by means of a CCD video camera with or without an image intensifier and stored electronically in computer memory, and/or magnetic or optical storage media such as CD-ROM or video tape. Other means of image capture and storage may also be employed. Electronically captured images can be evaluated utilizing image analysis methods that are well known to those skilled in the art, see Current Protocols in Cytometry (1997) or Digital Image Processing: PIKS Inside (2001) for general methodology. For example, differentiation between cells in which the chromatin has and has not been digested by DNAse and fluorescently stained may be accomplished by utilizing an adaptive thresholding method to segment the image into regions exhibiting pixel intensities above (putative nuclei) and below a threshold value; and subsequently determining the size, shape, and mean of integrated pixel intensity of each above a threshold region. One of many possible suitable embodiments of such a method utilizes a DAGE MTI (Michigan City, Ind.) or a Photometrics (Tucson, Ariz.) cooled CCD camera to capture images of the specimen. Automatic image focusing is accomplished using the constrained iterative autofocus algorithm included in the VayTek Microtome image deconvolution software package (VayTek, Fairfield, Iowa). Regions of interest can be manually defined and the mean pixel signal levels within these regions can be determined using the Scanalytics EPLab image quantitation software (Scanalytics, Fairfax, Va.). This software can also be employed to perform routine image preparation operations including, but not limited to field flattening; background and “hot pixel” correction; and fixed and/or adaptive thresholding. More sophisticated methods that are known to those skilled in the art such as, but not limited to pixel tracking; morphological analysis; pattern matching; correlation and similar algorithmic image analysis methods may be employed as appropriate to specific applications of the present invention.

The presence of exogenous materials such as stains that are commonly utilized to facilitate the visibility of cells, cell constituents, and cell structures in transmitted light, reflected light, and fluorescence microscopic techniques can potentially interfere with the growth of cells. For this reason, certain embodiments utilize phase contrast or other similar imaging mode that does not require the use of such contrast enhancement agents to facilitate specimen visualization and/or imaging until such time as cell growth has been completed at which time the specimen is treated with a DNA binding dye, stain, or other reagent that selectively increases the contrast between the chromatin and the other materials in the specimen. For example, the fluorescent DNA binding dye ethidium bromide is specified in the following descriptions of preferred embodiments of the invention. Numerous additional suitable fluorescent, absorbing, and other types of contrast enhancement agents are known to those skilled in the art, see for example Molecular Probes: Handbook, updated Sep. 7, 2003, www.probes.com/handbook, which is incorporated herein by reference. Of these, certain fluorescent DNA binding dyes including, but not limited to dyes of the TO-PRO, YO-YO, YO-PRO and PO-PRO families, that bind specifically and stoicheometrically to DNA and that undergo a significant enhancement in fluorescence upon binding to DNA are particularly beneficial in those embodiments of the present invention wherein it is desired to quantitate the amount of DNA present.

Numerous other suitable methods of microscopic imaging, image capture, and image analysis are known to those skilled in the art. The methods identified herein are for exemplary purposes and do not in any way limit or constrain the scope of the present invention.

A preferred method for analyzing the staining and assessment of cellular DNA is by flow cytometry or laser scanning cytometry. In an even more preferred embodiment, cells that are stained with a quantitative DNA stain are subjected to flow cytometry. Flow cytometry can be performed with a fluorescent activated cell sorter (FACS) as known in the art. Exemplary FACS machines that can be used include FACS-Calibur (Becton Dickinson; Mountain View, Calif.) and a Coulter flow cytometer (Hialeah, Fla., USA) EPICS Elite®. Quantification can be performed using CellQuest (Becton Dickinson; Mountain View, Calif.), WinList (Verity Software House, Inc. Topsham, Me.), Multicycle software (Phoenix Flow Systems, San Diego, Calif. USA) and FACScan (Becton Dickinson, Mountain View, Calif.) software.

A flow cytometer measures the amount of light-emitting substance associated with each cell and other parameters and provides output in the form of, e.g., a histogram, dot plot, or fraction table. The amount of one light-emitting substance associated with each cell can be compared to other properties of that cell, such as the amount of another light-emitting substance to which the cell population has also been exposed, size, granularity, or inherent light-emission.

As sheath fluid containing cells passes through the laser, typically one-by-one, they are exposed to light of various wavelengths. Each particle detected by the cytometer is termed an “event.” The degree to which an event transmits or scatters some of the incident light provides a measure of the event's characteristics, e.g., associated light emitting substance. For example, the event may emit light of its own accord or may emit fluorescent light generated by a fluorescent substance introduced into the event. An example of such a substance is a fluorescent DNA stain. A fluorophore responds to incident light of a particular frequency by emitting light at a known frequency that is detected by, e.g., photomultiplier tubes (PMTs) of the cytometer. The intensity of the emitted or reflected light is measured and stored by the cytometer.

The cytometer compiles emission data into a histogram. The histogram may be reported in one-dimensional form. Alternatively, it may be combined with a histogram of emitted light resulting from other incident wavelengths. Such a combination is typically reported as a “dot plot,” in which events are plotted on a grid, and the axes of the grid correspond to the two parameters being measured. For example, events could be exposed to incident light of a particular wavelength and assayed for forward light scatter and for emission at another wavelength.

A cell population may be segregated based on their DNA content. A peak will occur at propidium iodide staining corresponding to the normal DNA content of cells. Peaks may also occur at higher multiples of the haploid number n, possibly corresponding to polyploid or mitotic cells. Peaks or above-background plateaus may also occur at propidium iodide staining levels that do not correspond to multiples of haploid number n. These events may correspond to cells that are sensitive to various DNA degrading agents. Gates may be formed to distinguish cells falling into various ranges of DNA content from cells with differing DNA content.

Other methods for identifying the DNA content of cells using a quantitative DNA stain and histochemistry. These techniques can also be combined with flow cytometric analysis. For example, certain cells can be separated out from other cells via flow cytometry. These cells can then be analyzed for DNA content using a non flow cytometric techniques.

V. Kits and Diagnostics

In various aspects of the invention, a kit is envisioned for diagnosis and/or detection of cellular invasiveness. In some embodiments, the present invention contemplates a diagnostic kit for detecting cellular invasiveness of cells in a human tissue sample or biopsy. The kit may comprise reagents capable of detecting matrix or cellular components for the identification, analysis, or detection of cellular invasiveness as described herein. Reagents of the kit may include at least one substrate having one or more regions of matrix materials, with variable thickness(es) within or between a region or regions, on the surface of or within the substrate, and any of the following: culture medium or media, buffers, immunohistochemical reagents, microscopy reagents, antibodies or antigens, or a combination thereof.

In some embodiments, the kit may also comprise a suitable container means, which is a container that will not react with components of the kit, such as an eppendorf tube, an assay plate, a syringe, or a tube.

The kit may further include an instruction sheet that outlines the procedural steps of the assay, and will follow substantially the same procedures as described herein or are known to those of ordinary skill.

### EXAMPLES

The following examples are included to demonstrate preferred embodiments of the invention. It should be appreciated by those of skill in the art that the techniques disclosed in the examples which follow represent techniques that function well in the practice of the invention, and thus can be considered to constitute preferred modes for its practice. However, those of skill in the art should, in light of the present disclosure, appreciate that many changes can be made in various embodiments disclosed and still obtain a like or similar result without departing from the spirit and scope of the invention.

### Example 1

### Device Fabrication—Discrete Heights

A device suitable for the practice of this invention may consist of one or more regions or zones of material of defined composition and thickness that is deposited or formed upon or within a substrate. In certain embodiments, such devices include multiple regions or zones of material deposited or formed upon or within a substrate. Each region may have the same or different composition and/or thickness as one or more other regions of material on the same substrate. In some instances it is desirable for the region(s) to abut one another. In other instances it is desirable for the regions to be spaced apart or divided by a barrier.

An example of such a process includes, but is not limited to a mask created using SU-8/250 negative resist (Microlithography Chemical Co.) that can be prepared as follows. Suitably cleaned substrates are dehydrated at 200° C. for 1 hr and cooled to room temperature in a dry atmosphere. The substrate is then mounted in the chuck of a wafer spinner; a suitable volume of SU-8 photoresist is statically dispensed in the center of the substrate and allowed to spread for 25 sec; and then spun for 15 sec at 850 RPM. The resist coated substrate is then soft baked for at least 6 hours at 95° C.; exposed through a negative photomask to UV light for 11 seconds at a flux of 20 J/cm²; hard baked at 95° C. for 15 min and developed in SU-8 developer. Single layer masks of up to 300 microns in thickness can be prepared in this manner using this photoresist. Other photoresists or multiple applications of the same photoresist can be used to prepare thicker masks and ones in which the features are of various controlled depths.

### Example 2

### Device Fabrication—Gradient

In some instances it is convenient or desirable that the thickness of the matrix protein within a zone vary in a continuous manner between some minimum and maximum values. This is most conveniently accomplished by forming the zones of matrix on the surface of the substrate in accordance with any of the damascene procedures described above, but modifying the procedure such that the substrate is supported at an angle relative to the horizontal that is calculated to result in the desired thickness gradient during the denaturation baking step of the process. Alternatively, the desired thickness gradient can be formed into the substrate by casting, molding, embossing, engraving, etch; or hot stamping prior to applying the matrix protein.

### Example 3

### Determination of Cellular Invasiveness

A device consisting of two sets of two parallel lines of collagen matrix protein, each line being 10 microns by 100 microns in extent on 100 micron centers, was fabricated on a glass microscope coverslip by casting each line in an opening in a silicone rubber mask as described above. The thickness of the matrix material comprising each line was controlled by adjusting the amount of matrix protein solution dispensed into an opening in the mask. The two sets of parallel lines had matrix protein thicknesses of approximately 30 microns (thin matrix) and 200 microns (thick matrix), respectively, after annealing at 55° C. for one hour. After removal of the mask, the coverslip with inscribed lines of matrix protein was placed on the bottom of a plastic cell culture dish and covered with approximately 30 mL of DMEM cell culture medium supplemented with 10% fetal calf serum. A micropipette was used to seed one line in each set with non-invasive OCM-1a melanoma cells at a density of approximately 10,000 cells per line. In a similar manner, the second and third lines in each set were seeded at similar densities with M-619 moderately invasive melanoma cells and MUM-2B highly invasive/metastatic melanoma cells, respectively. Likewise, the adjacent interline spaces were seeded with OCM-1a, M-619 and MUM-2B cells. The substrate surface in these interline regions consists of serum proteins absorbed onto the substrate from the cell culture medium. The cells seeded onto the substrate were grown by incubation for 24 hours at 37° C. in an atmosphere consisting of 5% CO₂/balance air.

Phase contrast images of non-invasive OCM-1a cells on absorbed serum protein, thin matrix and thick matrix show that these cells form isolated small clusters under all three conditions. Phase contrast images of moderately invasive M-619 cells on absorbed serum protein, thin matrix, and thick matrix show that these cells form isolated cell clusters on absorbed serum protein and cord-like structures on both thin and thick matrix. Phase contrast images of highly invasive MUM-2B cells on absorbed serum protein, thin matrix and thick matrix show that the MUM-2B cells grown on absorbed serum protein formed isolated cell clusters of substantially greater size than is observed for either the OCM-1a or M-619 cells grown under the same conditions. On thin matrix, the MUM-2B cells form networks of cord-like structures while, on thick matrix, these cells form tumor nests embedded in extensively remodeled matrix protein and exhibit the looping patterns associated with this type of structure.

A phase contrast image of MUM-2B cells at the boundary between substrate regions having absorbed serum proteins and having thick matrix as compared to a fluorescence image of this same region after the cells were: permeabilized with 0.1% Triton X-100 for 3 min; treated with DNase for 60 minutes; and stained with the nucleic acid stain ethidium bromide show that the chromatin of the highly aggressive MUM-2B cells on thick matrix remains intact after this treatment. The intact cells are indicated by large, roughly spherical, brightly stained cell nuclei while chromatin of those MUM-2B cells on absorbed serum protein is degraded to the point where only nucleoli (small, bright points of light in the image) remain. Under these conditions, the chromatin in normal cells, non-invasive OCM-1a cells, and moderately invasive M-619 cells is completely degraded independent of whether the cells were on matrix or on absorbed protein. Reducing the duration and stringency of the conditions used for the DNase digestion permits retention of the chromatin in approximate descending order from most stable to least stable of invasive cells on matrix, normal or non-invasive cells on matrix, invasive cells on absorbed protein and normal or non-invasive cells on absorbed protein.

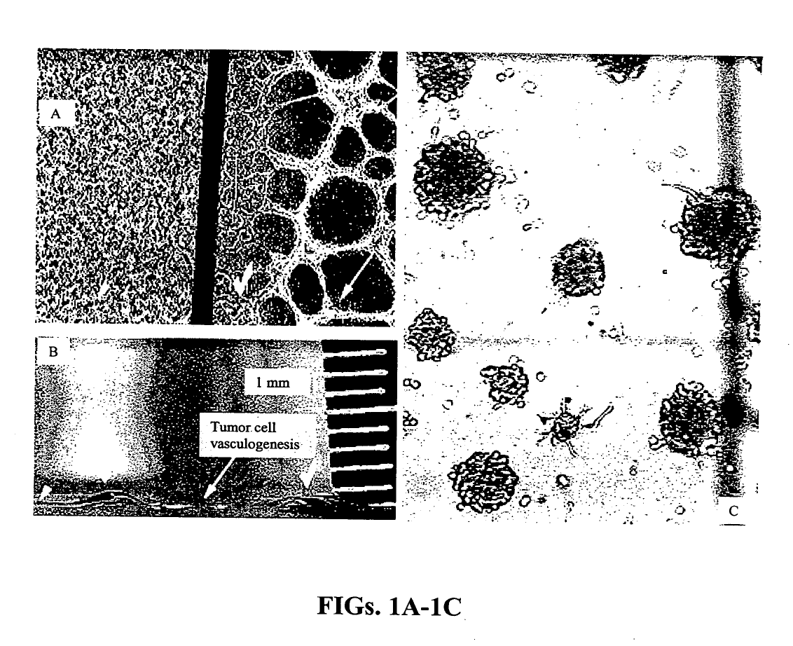

Also, FIG. 1A shows highly invasive MUM-2B cells growing on extracellular matrix protein. The thickness of the matrix protein varies from approximately 35 nm (absorbed protein) on the left (arrowhead mark) to approximately 1 mm on the right (long arrow mark). On matrix thicknesses of up to about 50 microns (to the left of the black line) the cells form random monolayer aggregates. In the matrix thickness range between about 50 and 150 microns (check mark), the cells form cordlike structures that mimic tumor vascularization. At thicknesses above about 150-250 microns, the cells form cylindrical or spheroidal tumor nests that are surrounded by remodeled matrix protein. FIG. 1B shows non-invasive OCM-1a cells growing on a similar matrix gradient (thickness increases to the right). These cells form discrete random aggregates at all thicknesses of matrix protein. Furthermore, FIG. 1C shows non-aggressive cells forming spheroids on any thickness of matrix.

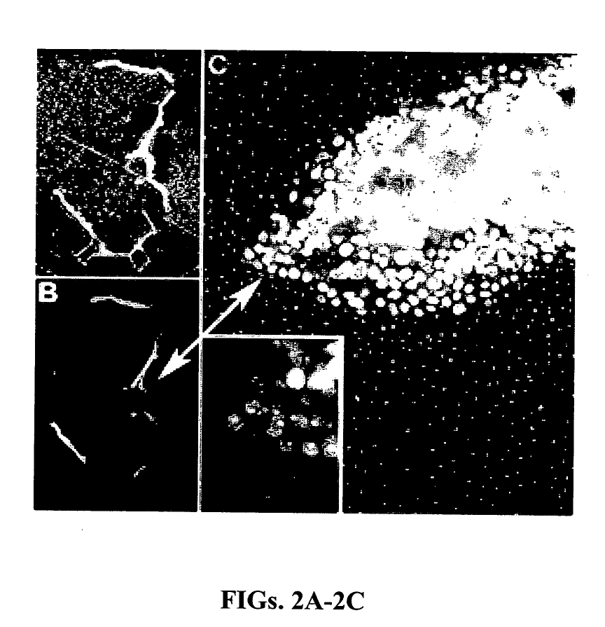

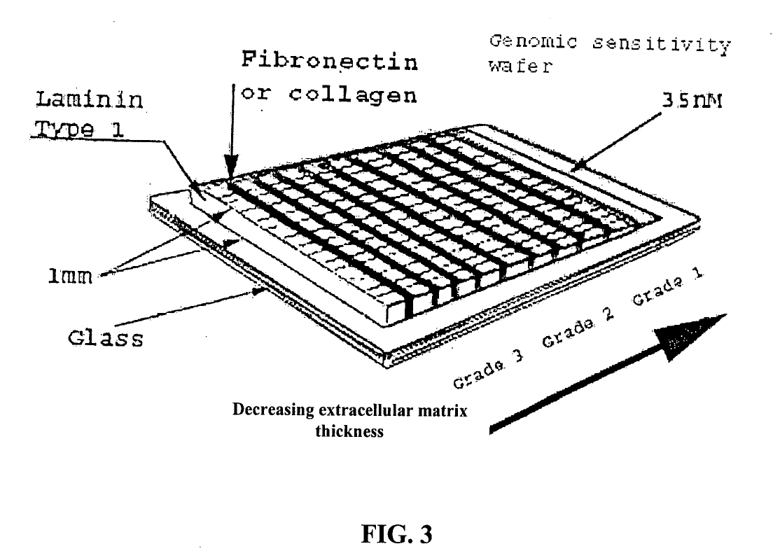

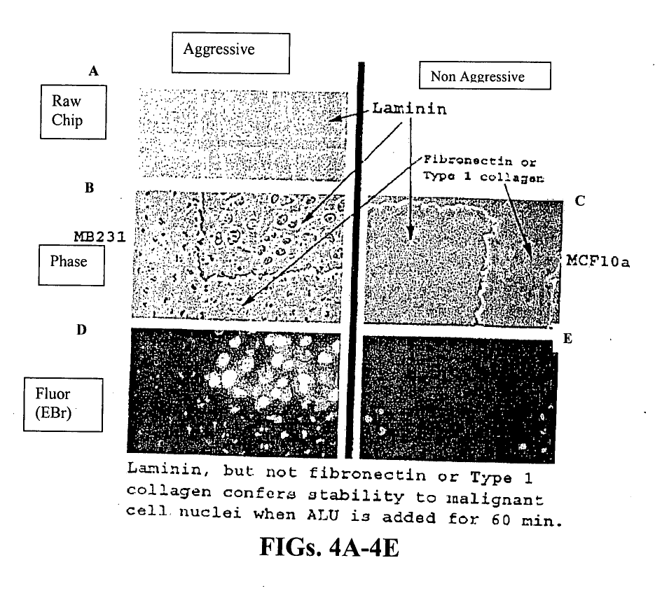

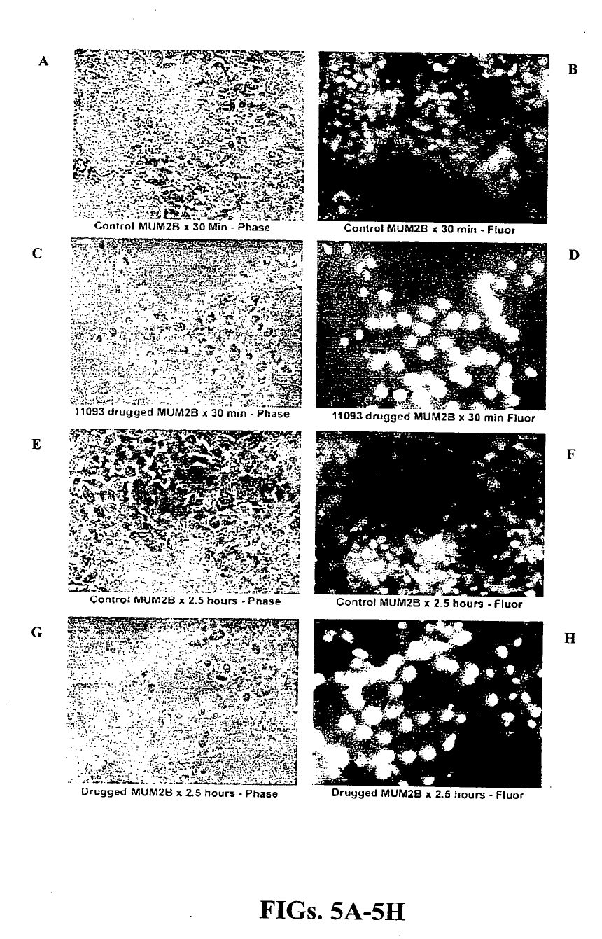

FIG. 2A shows a bright field image of laminin matrix protein deposited at about 100 microns thickness in the shape of the numeral “3” on a uniform layer of absorbed laminin protein about 35 mn thick. MFC-10A breast cancer cells were deposited uniformly over this entire area and digested with DNAase. FIG. 2B shows a fluorescence image of this same area after staining with the DNA stain ethidium bromide. The localization of the cellular DNA to the region of thickest matrix protein is clearly evident. FIGS. 2C and 2D show images of the central portion of the region shown in FIG. 2B at progressively higher magnifications. The cells growing on the thin matrix were unaffected by the action of DNAase. The small fluorescent objects in the area of absorbed matrix protein surrounding the cells are nucleoli that remained after the DNA in the cells was digested by the DNAase treatment.

### Example 4

### Detecting Invasive Cells in Mixed Cell Cultures

The presence of invasive and, therefore cancerous, cells in a cell or tissue specimen, such as might be obtained from a patient by biopsy or similar method, may be determined in the manner described above for determining the invasive potential of cells except that the cells seeded on the zones of cell matrix and absorbed serum protein present on the device consist of mixtures of cell types produced by the disaggregation of the cell or tissue specimen. At low cell densities where cell-cell contact between cells resulting from the growth of the cells initially seeded on the substrate is minimized, each cell type present in the mixture of cell types seeded on the substrate behaves largely independently of the other cell types that may be present. Thus, for example, moderately invasive cells will form cord-like structures on both thin and thick matrix while highly invasive cells will form networks of cords on thin matrix and tumor nests with associated looping patterns on thick matrix. This behavior permits differentiating normal and non-invasive cells in the specimen from invasive and thereby cancerous cells in the same specimen. This differentiation can be flirther perfected and the signal to noise ratio of the determination further improved by determining the susceptibility of the chromatin in the cells comprising the mixture in accordance to the DNase digestion procedure described previously. In particular, growth of the mixed cell types on thick matrix followed by DNase digestion of the chromatin and staining of the chromatin with a nucleic acid stain provides clear differentiation between invasive (cancerous) and normal cells.

Additional insight into the presence of invasive (cancerous) cells in a specimen including a mixture of cell types can be obtained by observing the behavior of cells when they are allowed to grow into contact with one another. On thick matrix, for example, normal cells and non-invasive cells can coexist in contact with one another. Furthermore, normal cells can penetrate layers or clusters of non-invasive cells, but non-invasive cells rarely penetrate layers or clusters of normal cells. Furthermore, normal cells do not coexist with invasive and particularly highly invasive cells on thick matrix. Specifically, normal cells are rapidly (typically less than one hour) lysed and killed when they come into contact with invasive cells. Invasive cells can further readily penetrate layers and clusters of normal cells, but normal cells cannot penetrate the structures formed by invasive cells. These behaviors are clearly evident from the cellular patterns generated when mixtures of cells are grown in accordance with the present invention and are especially evident when the signal to noise ratio of such patterns is improved by the DNase digestion and fluorescent staining of the cellular chromatin in the manner previously described.

An alternative implementation of this method is of utility in certain clinical instances. In one alternative implementation, a monolayer of normal cells corresponding to the normal cell types that comprise the type of tissue from which the clinical specimen is obtained is grown under the appropriate conditions on thin or thick matrix. An aliquot of mixed cell types obtained by the dispersal of the cells comprising the clinical specimen is then seeded at low cell density on top of the monolayer of normal cells and incubated as previously described. Invasive cells present in the dispersed clinical specimen will penetrate and lyse those underlying normal cells with which they are in contact resulting in pronounced, readily identified localized disruptions to the structure of the monolayer. The signal to noise ratio of such patterns is improved by the DNase digestion and fluorescent staining of the cellular chromatin in the manner previously described. Normal and non-invasive cells that may be present in the dispersed clinical specimen do not penetrate and lyse the layer of normal cells and, therefore, do not disrupt the structure of the monolayer.

### Example 5

### Metastatic Sites

Two exemplary devices, each including seven parallel lines of matrix protein with each line being approximately 10 microns wide by 100 microns long on 100 micron centers, were fabricated on a glass microscope coverslip by casting each line in an opening in a silicone rubber mask as described above. Each line thus formed consisted of a different matrix protein, specifically: fibronectin, laminin, collagen I, and matrix materials isolated from liver, bone, lung and thymic husk tissue. The thickness of the matrix material comprising each line was controlled by adjusting the amount of matrix protein solution dispensed into an opening in the mask. The lines had a matrix protein thickness of approximately 200 microns (thick matrix) after annealing at 55° C. for one hour. After removal of the mask, the coverslip with inscribed lines of matrix protein was placed on the bottom of a plastic cell culture dish and covered with approximately 30 mL of a culture medium suitable to the cell type to be evaluated. As examples, the culture medium used for melanoma cells was DMEM supplemented with 10% fetal calf serum, which was further supplemented with essential amino acids, epidermal growth factor, and insulin when used for the growth of breast cancer cells.

T4 breast cancer cells were seeded on each line of matrix protein on one device at a density of approximately 10,000 cells per line while MUM-2B melanoma cells were similarly seeded on the matrix protein lines of another device, each device being immersed in a culture medium appropriate to the cell type as indicated above. Both devices with seeded cells were incubated at 37° C. in a 5% CO₂/balance air atmosphere for 24 hours before the resulting cell growth patterns were evaluated.

Neither cell type formed tumor nests, looping patterns, or cord-like structures on fibronectin matrix. The T4 breast cancer cells produced well defined cellular patterns on laminin, bone, lung and thymic husk matrix materials, but did not produce defined structures other than aggregates on the collagen and liver matrix materials. The MUM-2B cells formed well defined cell growth patterns on all of the matrix materials except fibronectin with the patterns formed on liver matrix being the best developed. These observations are consistent with the known propensity of T4 breast cancer cells to initially metastasize to laminin-rich sites followed by secondary metastasis to lung and thymus, but not to liver and with the known propensity of MUM-2B cancer cells to initially metastasize to liver with subsequent metastasis to bone and other sites. Cells derived from other types of cancers similarly show preference for matrix proteins that are characteristic of the site of primary tumor formation and metastasis.

The metastatic preferences and proclivities of tumor cells can be further characterized by permeabilizing the cells grown on different types of matrix proteins with Triton X-100; treating the cellular chromatin with DNase; and staining the cells with a DNA stain such as ethidium bromide as previously described. The relative susceptibilities of the cells grown on the different matrix materials to chromatin degradation by DNase can be estimated by observing the effects of varying the concentration of DNase in the digestion reagent and the time for which the cells are exposed to DNase on the degree of chromatin degradation. In this manner it can be shown that the chromatin in T4 breast cancer cells on laminin matrix, MUM-2B cells on liver matrix, and prostate cancer cells on bone matrix are significantly more resistant to DNase digestion than is the chromatin of the same cells grown on other matrix materials.

An alternative device configuration that is sometimes preferable for the determination of metastatic preferences and proclivities is identical to that described above except that either the masking material used in the formation of the matrix protein zones is left in place or the matrix protein is embedded in the body substrate by methods previously described rather than being formed upon it. In either case, the exposed surface of the matrix material is approximately coplanar with the surrounding mask or substrate material. Such a configuration permits a single volume of cell suspension to contact the exposed surfaces of all matrix zones that are present on the substrate and is particularly convenient when the intend is to screen a mixed population of cells such as may be obtained by disaggregation of a biopsy specimen for the presence of invasive and metastatic cells. Devices of this configuration are used in the manners described above except that it is sometimes desirable to remove the residual cell suspension from the device after some settling period, but before initiation of incubation in order to limit the density of cells seeded on the matrix zones.

### Example 6

### Drug Evaluation

Exemplary devices of the invention may also find utility in the screening and evaluation of anti-cancer drugs in both clinical and drug discovery settings. In particular, embodiments of the invention are of use in screening a number of anti-cancer drugs for efficacy against multiple cancer types and for the determination of the most efficacious anti-cancer drug or combination of such drugs for the treatment of a patient. Such use is exemplified by the methods described below.

A device including four sets of two parallel lines of collagen or other selected matrix protein(s), each line being approximately 10 microns by 100 microns in extent on 100 micron centers, was fabricated on a glass microscope coverslip by casting each line in an opening in a silicone rubber mask as described above. The thickness of the matrix material comprising each line was controlled by adjusting the amount of matrix protein solution dispensed into an opening in the mask. One line in each set of parallel lines had a matrix protein thickness of approximately 30 microns (thin matrix) while the other line in the set had a thickness of about 200 microns (thick matrix) after annealing at 55° C. for one hour. After removal of the mask, the coverslip with inscribed lines of matrix protein(s) was placed on the bottom of a plastic cell culture dish and covered with approximately 30 mL of DMEM cell culture medium supplemented with 10% fetal calf serum.

A micropipette was used to seed both the thin and thick matrix lines in one set of lines with untreated MUM-2B cells at a density of approximately 10,000 cells per line and seed the exposed absorbed serum protein between these lines at an area proportional density (about 90,000 cells). In a similar manner the lines and intervening spaces comprising the second, third and fourth sets of lines were seeded with normal endothelial cells, MUM-2B cells that have been treated with polyamine 11157 according to the protocol established for such treatment; and normal endothelial cells treated with polyamine 11157 in an identical manner, respectively. In this example, the polyamine-treated MUM-2B cells constitute the experimental sample while the cells applied to the other sets of lines serve as controls. The number of sets of lines on a substrate can obviously be increased to accommodate replicate samples; additional drugs and treatment regimens; additional controls; and/or additional types of cancers so long as the culture medium employed is compatible with the growth of all cell types to be evaluated on that substrate. The cells seeded onto the substrate are grown by incubation for 24 hr (longer for some other cell types) at 37° C. in an atmosphere consisting of 5% CO₂/balance air.

The cellular patterns generated on the matrix lines and intervening spaces are examined by any of the methods previously described to determine the efficacy of a compound, drug or treatment. In the case of anti-cancer drugs, drug candidates and treatment regimes, efficacy is indicated by suppression of the formation of cord-like structures, tumor nests and looping patterns in the experimental sample relative to the untreated cancer cell control. Simultaneously, an efficacious treatment has no effect on the growth and behavior of the normal cell controls present on the same substrate. The ability of a drug or treatment to reduce the resistance of the chromatin in the abnormal cells to digestion by DNase while not affecting the behavior of the chromatin in the normal cell controls can also be an indicator of drug or compound efficacy. This method of screening anti-cancer drugs is particularly beneficial in that unlike the methods presently employed for this purpose, the inventive method directly addresses the known fact that cancer cells that have formed tumor nests are as much as 150 times more resistant to anti-cancer drugs than are the same cells grown under traditional culture conditions.

This same method can be employed in the identification of a drug, drug combination, or treatment regimen that may be particularly beneficial to a specific patient. This method is implemented in the same manner as described for drug screening except that the cancer cells employed are obtained by disaggregating a cancer containing biopsy specimen from the patient and the normal control cells are obtained by disaggregating normal cells obtained from the same tissue or organ.

This method may also be applied in an inverse manner in which the experimental cells are exposed to a material that is being evaluated to determine its ability to promote or potentiate cancerous transformations. The cells used for this purpose will typically be normal or minimally invasive phenotypes. A material that promotes or potentiates cancerous transformation causes some portion of these initial cells to exhibit morphological characteristics such as the formation of cords, cord networks and/or looping patterns that are associated with invasive cell phenotypes. This method may find utility in areas including, but not limited to occupational health, environmental testing and biological research.

### Example 7

### Drug Evaluation—Cytoplasts

Another novel aspect of the invention is that the morphologies of cells grown on matrix proteins are in many respects not controlled by gene expression and other processes that occur within the cell nucleus, but rather are controlled by processes and events within the cell cytoplasm. By virtue of this observation it becomes possible to unambiguously differentiate between, by way of example, the actions of a drug such as an anti-cancer drug that affect processes within a cell nucleus and those that affect cytoplasmic processes.

Unambiguous differentiation between cytoplasmic and nuclear processes requires that the nuclei be removed from the cells to be evaluated. Although this can be accomplished microsurgically, obtaining sufficient numbers of enucleated cells (cytoplasts) by this method is extremely labor intensive and time consuming. For this reason, a bulk method for cell enucleation has been developed. In this method, the cells to be enucleated are grown to near confluence on glass microscope coverslips coated with serum fibronectin. The coverslip is then placed cell-side down in a 50 mL Centrifuge tube containing 10 mg/mL of cytochalasin B and centrifuiged at 8,000×G for 30 min to force the cell nuclei to be displaced through the cell membrane. The cytoplasts thus formed remain attached to the coverslip and can either be recovered as a suspension or can be transferred directly to a layer of laminin or other matrix protein by contacting the cytoplasts to the matrix protein for a period of time.

Cytoplasts prepared in this manner largely recapitulate the morphology expressed by the parent cell when the cytoplasts are grown on matrix. Specifically, cytoplasts derived from normal, non-invasive, moderately invasive, and highly invasive cells form discrete clusters when grown on serum proteins absorbed onto a substrate. When these same cytoplasts are grown on, for example, thick matrix, those derived from normal and non-invasive cells form isolated clusters (or cobblestone monolayers in the case of normal endothelial cells) while cytoplasts derived from invasive phenotypes form cord-like structures on thick matrix. Cytoplasts derived from highly invasive cells have not, as yet, been induced to form tumor nests and looping patterns on thick matrix. Cytoplasts can be directly substituted for or used differentially in conjunction with intact cells in the above described drug evaluation tests for the purpose of differentiating between the nuclear and cytoplasmic effects of the drugs being evaluated.

### Example 8

### Slide-Based Methods

Certain embodiments of the invention include a slide-based test. One example of a slide based test includes obtaining a test sample, (e.g., scraping cells off of their culture plates); suspending the sample in phosphate buffered saline (PBS); administering a drop of the cell suspension on a laminin coated slide. The sample administered to the coated slide is allowed to air dry (about 15 minutes). The dried slide is subjected to digestion with ALU (about 15 minutes). After digestion the slide is stained with ethidium bromide (1 minute) and imaged under fluorescence. Aside from being fast, this method is an improvement on standard “smear” methods (like Pap tests) in that it is insensitive to the air-drying artifacts that cause major problems with the standard methods and necessitate using preservatives and fixatives. An alternative sampling technique includes depositing cells by contacting the slide with a tissue block. The exfoliated cells (which are the cells of interest) stick to the slide while most of the other cells do not.

### Example 9

### Mechanically Damaged Fibroblasts

Another example of using the compositions, methods and devices described herein include using mechanically damaged fibroblasts as a sample. A significant percentage of the damaged cells have been observed to yield a distinctive “ring” pattern when digested and stained. The damaged fibroblasts may be used as a model for “reactive” and “repairative” cells. The damaged fibroblasts are otherwise normal cells that are recovering from physical damage, infection or the like. It is difficult to distinguish these damaged fibroblasts or cells similar to damaged fibroblasts from abnormal cells in standard microscopy and immunochemical tests. This inability to distinguish between an essentially normal cell undergoing a repairative process and an abnormal cell is a common cause of false positives. In various embodiments of the invention the reactive and repairative cells are clearly differentiated from abnormal cells

### Example 10

### Inverted Format

In yet another aspect of the invention, the slide based format may be “inverted.” That is a cell that would demonstrate sensitivity to the treatments described herein can be treated in such a way that a sensitive cell will be insensitive to treatment and an insensitive cell will be sensitive to treatment. Thus, the result of the method is inverted. Inversion of the test can be produced by adding a thiol reagent such as DTT to the treatment solution. In other words, if the regular test is configured such that abnormal cells remain intact and normal cells are degraded, adding DTT reverses this result such that normal cells remain intact and the abnormal cells are degraded.

### Example 11

### MSP I Digestion

Methods

Human fibroblasts, poorly invasive human melanoma cells (OCM-1), and highly invasive human melanoma cells (MUM-2B) were scraped from their plastic cell culture flask bottoms with a rubber policeman to avoid disrupting their chromatin structure with EGTA, or their glycocalyces with trypsin. A 25 μL drop of each cell slurry was placed on a glass slide, and incubated for 30 minutes to an hour, until the drops-containing cells completely dried. Then, 0.5 μL of MSP I was added to a 25 μL drop of DMEM or PBS, and the 25 μL drop was placed onto the dried cell blots, and the slide was then placed in a 37° C. incubator in a sealed humidified chamber for 24 hours. After digestion, MSP I was removed and replaced with ethidium bromide, visualized under an epi-fluorescence microscope, and the blots were photographed.

Results

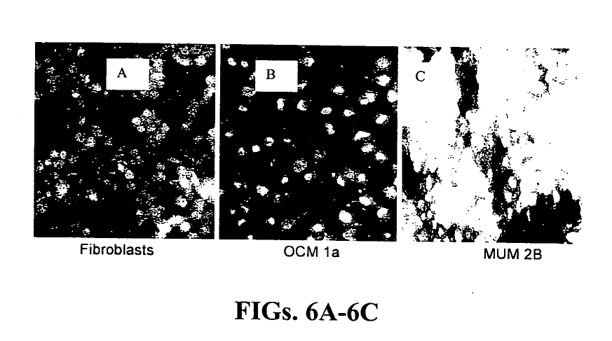

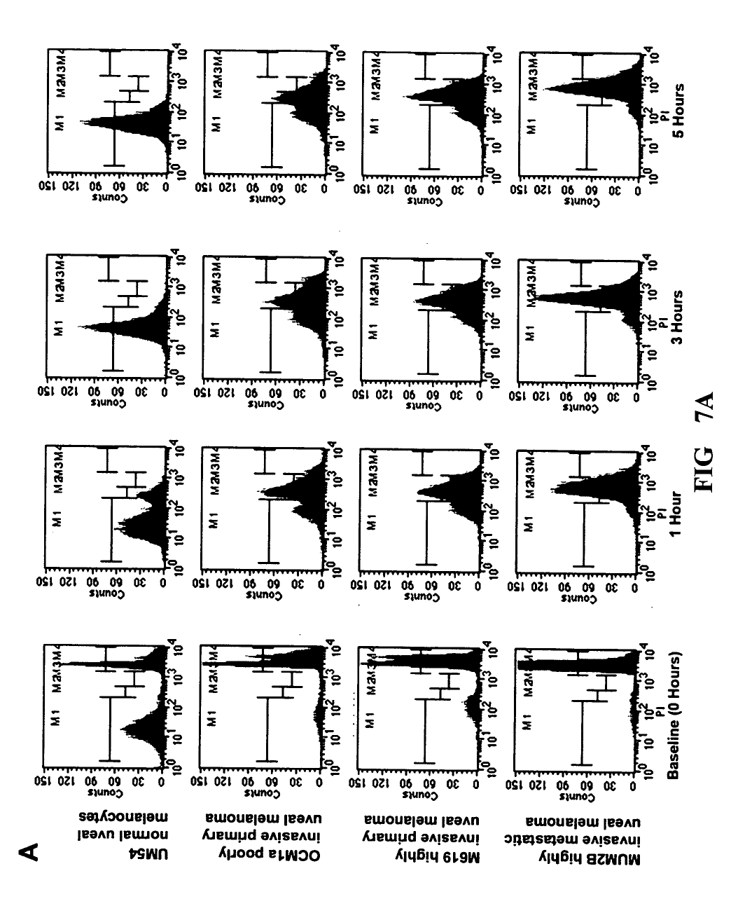

Whether incubated for 1, 2, 4, 5, 6, and 24 hours in MSP I, sequestration from digestion with MSP I appeared to increase with increasingly invasive cell behavior. Normal stromal cells, such as fibroblasts, were digested to a greater degree as compared to poorly invasive cells or to highly invasive cells. Cells are typically obtained mechanically, because trypsinization of cells in a trypsin-EGTA solution generated non-specific and sometimes completely refractory sensitivities to the enzyme(s). When trypsin EGTA was employed, for example, cell chromatin, regardless of the cell type, was much more stable to digestion with all restriction enzymes compared to mechanically isolated cells. A majority of the time cells demonstrated a gradation of sensitivity with normal cells being most sensitive, poorly invasive cells less sensitive, and highly invasive cell most refractory, if not completely refractory to digestion with MSP I, as well as other restriction enzymes (FIGS. 6A-6C).

The scraping of cells from flasks, rather than EGTA-trypsinizing them from flasks, typically serves two purposes: 1) anticipation of the MSP I digestion being employed in a translational setting, in which proteases such as trypsin are normally not, and could not be used to obtain cells from a human patient, and 2) avoidance of the reagent, EGTA, which is commonly employed to accelerate cell dissociation from tissues or tissue culture flasks. In addition, the hypothesis that withdrawal of essential ions such as magnesium with EGTA or EDTA disrupts adhesion receptors specifically, has been shown to be simplistic, due to the fact that these chelators have profound effects upon chromatin organization and structure (Maniotis et al., 1997). Experiments employing EGTA-trypsin as the means of cell isolation have shown that sensitivities to restriction enzymes among different cell types is radically more stable to digestion, probably because of the effects of EGTA, and differences in cell aggregation due to trypsin-induced clumping. Therefore, to enhance potential clinical utility, and to avoid altering chromatin organization MSP I was employed without protease digestion or the presence of EGTA but instead, simply mechanically removed cells from their environment as they would be removed from a patient.

The fact that MSP I digestion could differentiate among nuclei of cells belonging to normal, poorly invasive and highly invasive cells, suggests that methylation of higher order structure is important, and may be the key factor in regulating a cell's cancerous or non-cancerous state, as well as a cell's pattern of methylation. Because digestion was cell type specific, rather than gene-type specific, the assay potentially can discriminate between normal, lowly and highly invasive cells, from sporadic tumors (99%) where linkage groups are unknown, and which familial linkage is not established, as well as the familial tumors (1%) where suspected oncogenes (p53, p21, retinoblastoma (rb), and the like) are thought to play some causative role.

### Example 12

### Assessing Drug Resistance of Invasive Tumor Cells

Exemplary devices of the present invention may also find utility in the assessment of the drug resistance of invasive tumor cells. Information of this type is relevant to the selection of therapeutic drugs for the treatment of cancers, for the evaluation of new chemical entities as anti-cancer drugs, and for other similar purposes. Invasive tumor cells are known to form “tumor nests” consisting of tumor cells embedded in highly remodeled extracellular matrix protein, the entire structure being laced with perfusion channels that superficially resemble, but are not blood vessels. The presence of such structures in a tumor is strongly correlated with poor prognosis, resistance to anti-cancer drugs, and a high mortality rate.

Invasive tumor cells grown on thick layers of extracellular matrix protein form tumor nests in vitro and can be used as model systems for the evaluation of the efficacy of anti-cancer drugs against similar structures in vivo. Fluorescence microscopy images of a fluorescent dye Texas Red microinjected into a sinusoid of a tumor nest will typically show the movement of the dye into the perfusion channels between clumps of tumor cells in the tumor nest. This movement continues until the tumor nest is permeated by the dye. This same pattern of perfusion is evident when neutral fluorescent dyes are applied to tissues or layers of matrix proteins containing tumor nests rather than being injected into the nests. Similar measurements made using fluorescently labeled dextran conjugates indicate that neutral molecules of up to at least 2,000,000 molecular weight can enter into and accumulate within these perfusion channels and adjacent cell clusters.

Tumor cells, even ones of highly invasive types, do not form tumor nests when grown on thin layers of extracellular matrix protein. Rather, they form clusters or sheets of cells with no evidence of the formation of perfusion channels. The nuclei of tumor cells grown on thin matrix are rapidly and efficiently labeled by fluorescent DNA-binding dyes such as bisbenzimide or the anti-neoplastic drug doxorubicin. When the same types of invasive tumor cells are grown under conditions in which they form tumor nests and the nests are exposed to DNA-binding agents such as bisbenzimide and doxorubicin in the manner described above, only the nuclei of cells on the peripheries of the tumor cell clusters forming the nests are labeled by the dye even after prolonged exposure. Invasive tumor cells grown in vitro on thick matrix and stained by the addition of bisbenzimide to the culture medium show staining restricted to the nuclei of the cells on the peripheries of the individual cell clusters forming the tumor nest. These and other similar experiments reveal that polar agents such as bisbenzimide and doxorubicin are systematically excluded from the cells comprising tumor nests while these cells are accessible to neutral molecules of similar molecular weights.

The devices described in examples 1, 2, 3, and 5 (above) and alternative forms and formats such as microtiter plates containing matrix protein(s) of the appropriate thicknesses and compositions in its wells may beneficially be employed in the in vitro assessment of the efficacy of therapeutic agents against refractory invasive cancers. It is convenient to juxtapose layers of thin and thick matrix in such devices such that the cells growing on thin matrix serve as procedural controls for the cells growing on thick matrix. In all cases, cancer cells from a patient or other source are grown on thin and thick layers of extracellular matrix to the point that tumor nests are formed by the cells on the thick matrix. The cells are then exposed to the therapeutic agent, typically by addition of the agent to the cell growth medium, and the results of such exposure observed. An effective agent will label all of the cells grown on thin matrix and will penetrate into the clusters of cells forming the tumor nests on thick matrix. Efficacy correlates with increasing penetration. Further evidence of efficacy may be obtained by observation of tumor nests for signs of necrosis and/or apoptosis upon prolonged exposure to the therapeutic agent. Additional indications of efficacy may be obtained by exposing the cells treated with the therapeutic agent to nucleases such as ALU or DNAase in the manner previously described.

### Example 13

### Identification of Targers for Therapeutic Intervention

The devices and methods of the present invention may further be employed in the identification of potential targets for therapeutic intervention in the treatment of cancers and in the design, development, and evaluation of therapeutic agents directed against such targets. As has been exemplified in preceding examples, the susceptibility of the chromatin in invasive cells to degradation by agents such as ALU and the resistance of such cells to chemotherapeutic agents depends upon the composition and thickness of the extracellular matrix protein(s) upon which the cells are grown. Such characteristics reflect in certain measure the genetic composition and gene transcription activities within the cells, which, in turn, suggest targets for therapeutic intervention.

By way of example, highly invasive MUM-2B melanoma cells were grown on thin and thick matrix using devices such as described in preceding examples 1, 2 and 3. For the purposes of this example, it is preferred, but not required, that the zones of thin and thick matrix and the cells grown thereon are in sufficiently close proximity to each other to ensure that all cells grow in an environment that is identical except for the thickness of the matrix protein or other variable to be evaluated. The cells grown on thin and thick matrix are then separately harvested. Harvesting is preferably performed mechanically in order to avoid artifacts that may accompany the use of chemical methods such as treatment with chelating agents such as EDTA or EGTA or with proteolytic enzymes such as trypsin that are commonly employed for this purpose. The cells harvested from thin and thick matrix are then separately prepared for application to “gene array” chips such as an Affymetrix II Microarray (Affymetrix) in accordance with the procedures defined by the manufacturer of the chip. The data obtained from these chips is then analyzed, preferably using a paired T-test, correlation analyses or similar methods, to identify the specific genes for which expression differs between cells grown on thin and thick matrix.

In one specific example, approximately 1044 genes are differentially expressed between MUM-2B cells grown on thin matrix and those grown on thick matrix. MUM-2B cells grown on thin matrix differentially express genes for a variety of proteins including, but not limited to: multiple types of cyclins; several histones; thrombospondin; matrix proteins such as laminin, collagen and fibronectin; and the corresponding cell surface adhesion receptors for these matrix proteins. The expression of these proteins is suppressed in MUM-2B cells grown under identical conditions on thick matrix. Conversely, the MUM-2B cells grown on thick matrix express genes including, but not limited to P21, SPP1, osteopontin, BPAG and thrombomodulin that are not significantly expressed in the cells grown on thin matrix. In other words, the differential gene expression pattern presented by highly invasive MUM-2B cells grown on thin matrix is a pattern that is consistent with that expected from rapidly proliferating migratory (invasive) cells while that presented by the same cells grown on thick matrix corresponds to the pattern that is consistent with that expected from fully differentiated senescent cells. Moving the cells from thick matrix to thin causes them to revert from the senescent to the proliferative-invasive gene expression pattern and conversely.

Data from numerous ancillary sources can be correlated with the differential gene expressions produced by means of this invention to identify the pathways that are activated or suppressed and to identify the regulatory feedback loops between them. Such analyses can also lead to the identification of previously unknown genes and gene functions. By way of a limited example, the SPP1 gene encodes the protein osteopontin, a phospho-protein that is involved in multiple biological functions including bone growth and hemostasis. The expression SPP1 is suppressed in MUM2B cells grown on thin matrix (invasive state) and substantially elevated in MUM2B cells grown on thick matrix (senescent state). Thrombomodulin, a multi-functional protein that among its other functions participates in the thrombin-mediated cleavage of osteopontin, is up regulated in cells grown on thin matrix.

Cleaved osteopontin and thrombospondin are known to bind to the CD44 integrin receptor of cancer cells from many types of tumors and appear to reduce the adhesion between cells and the extracellular matrix. It is further known that the expression of ankyrin, which links the CD44 receptor to actin filaments of the cytoskeleton, and BPAG, which forms links between actin filaments and intermediate filaments, are elevated in cells grown on thin matrix. Other data shows that the CD44 receptor is mechanically linked to the nuclear pores via the actin filaments and other cytoskeletal components and that mechanical forces exerted on the CD44 receptor can “switch” the chromatin in the nucleus between the senescent and invasive phenotypes. The net effect of these events is the down regulation of the invasive potential of the cell. Cleavage of osteopontin by thrombomodulin reverses this down regulation. This simple illustrative cyclic pathway suggests that invasive cells can reversibly switch between invasive and senescent phenotypes in response to external conditions and implies that disruption of any of several steps in this pathway can lock the cell into one or the other phenotype.

The traditional “tactical” approach to cancer therapies is based upon the selective destruction of cancer cells by the disruption of specific pathways and/or molecular targets. This invention provides a means of identifying such tactical targets and for evaluating and validating agents developed against them. The illustrative pathway described above, even though incompletely described, is yet presented in sufficient detail to also suggest certain “strategic” therapeutic alternative and complementary methods. Practice of this invention in the manner described in example 6 and alternatives thereof permits demonstration that the response of cancer cells to a therapeutic agent can differ significantly depending upon whether the cancer cells are in their invasive or senescent phenotypic form. One strategic alternative suggested by this invention is the use of a therapeutic agent that locks cancer cells into the phenotype, typically the invasive phenotype, that is most sensitive to a second therapeutic agent. Another strategic alternative suggested by this invention and particularly relevant to highly aggressive, rapidly migrating cancers is treatment with a therapeutic agent that locks the cells into the senescent phenotype thereby effectively converting an acute disease state into a chronic disease state and providing additional time for the mounting of an effective treatment. Similar strategic treatment may prove to be a useful adjunct to the surgical removal of solid tumors in that, prior to surgery, the cancer may be placed into a senescent state to minimize the shedding of invasive cells. Furthermore, the efficacy of post-surgical chemotherapy may be augmented by switching the cancer cells into the invasive and more drug sensitive state during chemotherapy and then switching them to the senescent state post-chemotherapy to suppress metastasis.

In a similar manner, the device of example 5 can be used to identify specific molecular and regulatory targets that may be useful in the specific suppression of metastasis. In such cases, one zone of the device is comprised of the matrix protein composition found in a primary tumor while the composition of another zone corresponds to that of the extracellular matrix found in a tissue to which the tumor may metastasize. Differential gene expression modulated by the two types of matrix can be used to identify potential therapeutic targets and agents. These investigations can be further refined if the device is modified such that the zones of different matrix composition and/or thickness are isolated from one another in a manner that allows altering additional parameters such as gas tensions, pH, and the mixtures of cytokines present to more accurately reflect the respective environments in the tissues involved.

As described in conjunction with previous examples, these devices may further be employed to assess the efficacy of therapeutic agents developed in accord with this example.

### Example 14

### Reference Material for the Evaluation of Anti-Cancer Agents

The National Cancer Institute and other organizations prepare, make available and/or use standardized panels of tumor tissues and/or cells against which the efficacy of potential anti-cancer agents can be assessed. Some such panels are comprised of living tissues excised from patient tumors while others are comprised of cultured tumor cells grown on a substrate or in suspension. As is evidenced in the preceding examples, the behavior of tumor cells is strongly influenced by the conditions, particularly conditions related to extracellular matrix, under which they are growing. This environmental sensitivity is neither contemplated nor addressed in tumor panels as currently embodied. Improved tumor panels can be constructed by growing tumor cells of the requisite type or types on devices such as previously described in examples 1, 2, 3, and 5, wherein the compositions and thicknesses of the matrix proteins upon which the cells are grown are controlled in the manners previously set forth. Such improved panels are more representative of the environments in which in vivo tumor growth occurs and therefore provide for more realistic assessment of the efficacy of anti-cancer agents. These improved panels provide a more defined and controlled environment than do panels comprised of tumor tissues and therefore facilitate comparative assessments. By way of example, tumor panels are often prepared by growing tumor cells on a substrate comprised of absorbed serum proteins. This substrate corresponds to the “thin matrix” condition described previously and is not supportive of the formation of tumor nests and other manifestations of invasive cells seen on thicker matrix layers. As has been seen in previous examples, invasive tumor cells grown on thin matrix are more susceptible to the action of anti-cancer agents than are the same cells grown on thick matrix. Thus the apparent efficacy of agents tested against invasive cells grown on thin matrix will be artifactually elevated. Conversely, as also seen in the preceding examples, the invasive behavior of tumors grown on thick matrix can be less than that of the same cells grown on thin matrix. This can mask the efficacy of the agent being tested. More accurate testing can be performed if cells grown on both thin and thick matrix are employed as reference materials in the manner embodied in this invention.

### Example 15

### Detection of Invasive Cells by Flow Cytometry

The methods described in the preceding examples of the present invention for the detection of invasive cells require that the cells are in contact with a substrate, typically a layer of extracellular matrix protein, prior to the treatment of the cells with a chromatin-degrading agent. This step can be inconvenient in a clinical setting. The cells of hematological cancers are typically collected as suspensions of cells in a fluid medium such as blood or lymphatic fluid. Similarly, certain methods such as fine needle aspiration (FNA) that are in common clinical use for the initial collection of the specimens from solid tumors result in the formation of a suspension of the collected cells in a fluid medium. Furthermore, the dispersion of cells into a fluid medium is an intrinsic element in the process of preparing monolayer preparations on microscope slides and in preparing specimens for evaluation by tissue culture and similar methods. For this reason it is convenient to be able to practice the present invention in a manner that directly utilizes specimen cells in fluid suspension rather than requiring that the cells be first be transferred to a solid substrate. The utilization of suspended cells in the practice of this invention in a manner that is analogous to the methods of examples 3 and 4 above is exemplified as follows.

Cultured cells of differing degrees of invasiveness are utilized for illustrative purposes in this example. Suspensions of cells from patient specimens may similarly be employed. The cultured cell lines employed in this example are: WI-38 fibroblasts (normal cells); OCM1 (a poorly invasive a primary uveal melanoma); M619 (a highly invasive primary uveal melanoma); and MUM2B (a highly invasive metastatic uveal melanoma). All cells were grown in monolayer culture according to well-known standard methods; mechanically harvested into DMEM medium and pelleted by centrifugation at 1400 RPM for 5 minutes in a desktop centrifuge. Mechanical harvesting of these cultured cells is used in preference to the use of agents such as trypsin and EGTA for the release of the cells from their substrates. This avoids the potential disruption of chromatin structure that occurs with EGTA and the disruption of the glycocalyces as is caused by trypsin.

The cellular pellet was re-suspended in 0.1% Triton X-100; incubated for 1 minute at room temperature; spun down again at 1400 rpm for 5 minutes; and resuspended in DMEM. Propidium iodide (PI; 10 μl/ml; Molecular Probes, Eugene, Oreg.) was added to an aliquot of this suspension. 0.5 μl of Alu I restriction enzyme in 40 μl of DMEM was added to the remaining cell suspension and the preparation was incubated at 37° C. Aliquots of this mixture were taken for evaluation at 0 (baseline), 1, 3, and 5 hours after the addition of ALU. Propidium iodide was added to each of these digested samples. The resulting digested and stained cell suspensions were analyzed according to standard methods using a FACS Calibur flow cytometer (BD Bioscience, San Jose, Calif.) equipped with 488 nm laser excitation, detectors for forward and side scatter, and 520, 575, and 675 nm′ detectors for fluorescence signals. 10,000 cells were counted and the results were analyzed with FACS dot-plots and histograms. CellQuest software (BD Bioscience) was used for statistical analyses.

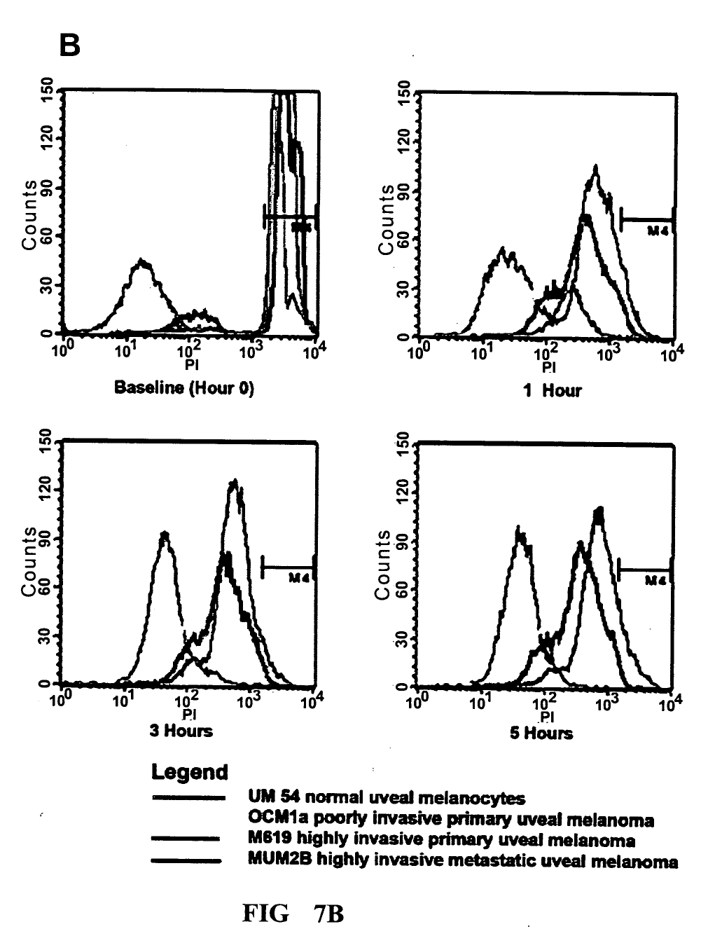

Propidium iodide is a stoicheometric DNA fluorescent staining agent thus allowing the DNA content of the cells being evaluated to be determined from the fluorescent signal intensity as measured by flow cytometry. The aliquot of cell suspension that was treated with PI after permeabilization, but not digested with ALU serves as a reference for the amount of DNA present in each of the cell preparations prior to the start of treatment. FIG. 7 shows the flow cytometer fluorescence intensity histogram plots measured for each cell line at 1, 3 and, 5 hours exposure to Alu I restriction enzyme followed by staining with PI.

A reduction in PI signal for UM54 normal uveal melanocytes was detected at one hour, with further decreases at 3 and 5 hours. By five hours, a significant component of the baseline signal decreased below the limits of the instrument's detection threshold (FIG. 7a, top row). This indicates a significant degradation of the DNA in normal uveal melanocytes. Poorly invasive OCM1a melanoma cells exhibited a similar reduction in the PI signal after 1 hour Alu I enzyme digestion, but thereafter, the signal intensity did not decrease significantly (FIG. 7, 2nd row).

Unlike the UM54 normal uveal melanocytes and the poorly invasive OCM1a melanoma cells, highly invasive M619 or MUM2B melanoma cells showed no significant loss of PI signal at one hour Alu I digestion (FIG. 7a, bottom two rows). However, the PI signal from the highly invasive primary M619 melanoma cells had decreased by 3 hours, while the signal for the highly invasive metastatic MUM2B melanoma cells was not significantly different from the baseline signal even at 5 hours. Therefore, on the basis of measuring the PI signal after exposure of the permeabilized cells to Alu I restriction enzyme for different periods of time, it is possible to discriminate between each of the four cell lines and thereby to utilize flow cytometry for the detection and classification of invasive cells (FIG. 7B).

### Example 16

### Drug Evaluation using Nuclei

Cells adherent to a 12 mm diameter glass cover-slip are prepared as described, above. The cover slip with adhered cells is placed cell-side down in a 50 cc conical centrifuge tube containing 5 cc of 10 mg/ml cytochalasin B in normal growth medium such that the edge of the cover-slip seats against the conical walls of the tube and the plane of the cover-slip is perpendicular to the long axis of the tube. Centrifugation at 1400 RPM for 5 minutes results in the cell nuclei being displaced through the cell membranes and collecting as a pellet in the bottom of the centrifuge tube. The enucleated cells remain attached to the cover-slip.

The collected cell nuclei are washed in DMEM and permeabilized by treatment with a 0.1% solution of the detergent Triton X-100 in DMEM for two minutes before being treated with ALU or DNAase. As described above, ALU selectively degrades the chromatin in nuclei from normal and non-invasive cells. Furthermore, treatment of the permeabilized nuclei with 100 units of DNAase in DMEM for 30-60 minutes digests the chromatin of the nuclei from normal and non-invasive cells while leaving the chromatin in nuclei from invasive cells largely intact. Other data related to example 13, but not herein described suggest that changes in the cytoskeleton which may be detectable by light scattering can influence the state of the chromatin in the cell nucleus. The displacement of the nucleus through the cell membrane that occurs in the present method appears to be sufficiently rapid as to permit differentiation between nuclear and cytoplasmic factors without significant levels of confounding interactive effects.

The descriptions of particular devices and methods embodied above are intended to be representative of and not limiting to the present invention. Although the devices and methods of this invention have been described in terms of preferred embodiments, it will be apparent to those skilled in the art that alternative implementations, compositions and/or methods herein described can be made without departing from the concept, spirit and scope of the invention. Specifically, it will be apparent that the devices herein described may be implemented by alternative means and that the compositions and conditions described herein may be altered for compatibility with specific cell and specimen types while still achieving the same or similar results as described herein. All such similar substitutes and modifications apparent to those skilled in the art are deemed to be within the scope and spirit of the of the invention as defined by the appended claims.

### References

The following references, to the extent that they provide exemplary procedural or other details supplementary to those set forth herein, are specifically incorporated herein by reference.
U.S. Pat. No. 5,162,114
U.S. Pat. No. 5,830,708
U.S. Pat. No. 6,372,494
U.S. patent application No. 60/427,646
Bojanowski et al., J. Cellular Biochem. Vol. 69:127-142, 1998.
Chan et al., Gut 52:502-506, 2003.
Current Protocols in Cell Biology, John Wiley & Sons, Inc., Bonifacino et al., (Eds.), 0-471-24108-3, 2001.
Current Protocols in Cytometry, Robinson et al. (Eds.), Wiley & Sons, 1997.
Digital Image Processing: PIKS Inside, 3rd Edition, Pratt (Ed.), ISBN: 0-471-37407-5, 2001.
Freshney, In: Culture of Animal Cells, 4^{th} Ed., Wiley-Liss, Inc., NY, 1-600, 2001.
Goessl et al., Prostate Cancer Prostatic Dis. Volume 3, Supplement 1, Pages S17-S17, 2000.
Maniotis et al., J. Cellular Biochem. Vol 65: 114-130, 1997
Molecular Probes: Handbook, probes.com/handbook, 2003.
Murphy, In: Fundamentals of Light Microscopy and Electronic Imaging, Wiley-Liss, 1-360, 2001.
Toyooka et al., Cancer Research 62, 3382-3386, Jun. 15, 2002.
Ueki et al., Oncogene. Mar. 27, 2002;21(13):2114-7, 2002.
Wong et al., Molecular Oncology, Markers, Clinical Correlates, 2003.

1-51. (canceled)

52. A method for determining the invasive potential of cells comprising:
a) depositing the cells to be evaluated on a substrate having one or more cellular growth regions comprised of a matrix material;
b) incubating the cells on one or more cellular growth region under conditions that allow for migration, growth, or migration and growth of the cells;
c) identifying cellular patterns that arise from the migration, growth, or migration and growth of the cells on one or more cellular growth regions; and
d) interpreting the cellular patterns to determine the invasive potential of the cells.

53. The method of claim 52, wherein at least a portion of one or more cellular growth regions are of a thickness sufficient to permit the cells to penetrate, remodel and become embedded in the matrix material.

54. The method of claim 52, further comprising treating the cells grown on one or more cellular growth regions with:
e) a cell permeabilizing agent;
f) an endonuclease, a nuclease, or both; and
g) a nucleic acid stain.

55. The method of claim 54, wherein the endonuclease is Alu, EcoRI, Mbo, Hind-1, or PST-1.

56. The method of claim 52, further comprising treating the cells grown on one or more cellular growth regions with:
e) an MSP I enzyme; and
f) a nucleic acid stain.

57. The method of claim 52, wherein the cells to be evaluated are a mixture of invasive and non-invasive cells.

58. The method of claim 57, wherein at least a portion of one or more regions of matrix material are of a thickness sufficient to permit the cells to penetrate, remodel and become embedded in the matrix material.

59. The method of claim 57, wherein the mixture of invasive and non-invasive cells are deposited upon a layer of normal cells grown on the one or more cellular growth regions.

60. The method of claim 59, wherein at least a portion of one or more cellular growth regions are of a thickness sufficient to permit the invasive cells to penetrate, remodel and become embedded in the matrix material.

61. The method of claim 52, wherein the matrix material is obtained or derived from one or more tissue or organ to which the invasive cells may migrate.

62. The method of claim 52, wherein the matrix material within a region is:
a) of a thickness A at which the cells to be evaluated do not penetrate and remodel the matrix material of the region;
b) of a thickness B at which the cells to be evaluated can penetrate and remodel the matrix material of the region, but which does not permit the cells to become embedded in the material;
c) of a thickness C at which the cells to be evaluated can penetrate, remodel and become embedded in the matrix material of the region; or d) a combination thereof.

63. The method of claim 52, wherein the matrix material comprising the cellular growth region is laminin, collagen, fibrinogen, fibronectin, cellular matrix material isolated from one or more biological tissues or a combinations thereof.

64. A method for screening a compound, drug or pharmaceutical composition for efficacy as an anti-cancer compound, drug or pharmaceutical composition comprising:
a) depositing cancerous or pre-cancerous cells on a substrate having one or more cellular growth regions comprised of a matrix material;
b) treating the cancerous or pre-cancerous cells with the compound, drug or pharmaceutical composition to be evaluated;
c) incubating the cancerous or pre-cancerous cells on one or more cellular growth regions under conditions that allow for migration, growth, or migration and growth of the cancerous or pre-cancerous cells;
d) identifying cellular patterns that arise from the migration, growth, or migration and growth of the cancerous or pre-cancerous cells on one or more cellular growth regions; and
e) interpreting the cellular patterns to determine the effects of the compound, drug or pharmaceutical composition on the cancerous or pre-cancerous cells.

65. The method of claim 64, wherein at least a portion of one or more cellular growth regions are of a thickness sufficient to permit the cells to penetrate, remodel and become embedded in the matrix material.

66. The method of claim 64, wherein the efficacy of an anti-cancer drug or pharmaceutical composition upon nucleated cells is evaluated.

67. The method of claim 64, wherein the efficacy of an anti-cancer drug or pharmaceutical composition upon enucleated cells (cytoplasts) is evaluated.

68. The method of claim 64, further comprising treating the cancerous or pre-cancerous cells with:
f) a cell permeabilizing agent;
g) an endonuclease, a nuclease, or both; and
h) a nucleic acid stain.

69. The method of claim 68, wherein the endonuclease is Alu, EcoRI, Mbo, Hind-1, or PST-1.

70. The method of claim 64, wherein the cells have been treated with the anti-cancer drug or pharmaceutical composition before being evaluated on a substrate having cellular growth regions comprised of a cellular matrix material.

71. A method for detecting compounds that initiate, promote or potentiate cancerous behavior in cells comprising:
a) depositing normal or non-invasive cells on a substrate having one or more cellular growth regions comprised of a matrix material; and
b) treating the cells with the compound to be evaluated;
c) incubating the cells on one or more cellular growth regions under conditions that allow for migration, growth, or migration and growth of the cells;
d) identifying cellular patterns that arise from the migration, growth, or migration and growth of the cells on the cellular growth regions;
and
e) interpreting the cellular patterns to determine whether the compound initiates, promotes and/or potentiates cancerous behavior in the cells.

72. The method of claim 71, wherein at least some portion of one or more cellular growth regions is of a thickness sufficient to permit the cells to penetrate, remodel and become embedded in the matrix material.

73. The method of claim 71, further comprising treating the cancerous or pre-cancerous cells with:
f) a cell permeabilizing agent;
g) an endonuclease, a nuclease, or both; and
h) a nucleic acid stain.

74. The method of claim 73, wherein the endonuclease is Alu, EcoRI, Mbo, Hind-1, or PST-1.

75. The method of claim 71, wherein the normal or non-invasive cells that have been treated with the compound to be evaluated before deposition on a substrate having one or more cellular growth regions comprised of a matrix material.

76. The method of claim 75, wherein the efficacy of an anti-cancer drug or pharmaceutical composition upon nucleated cells is evaluated.

77. The method of claim 75, wherein the efficacy of an anti-cancer drug or pharmaceutical composition upon enucleated cells (cytoplasts) is evaluated.
